(19) United States
(12) Patent Application Publication
(10) Pub. No.: US 2026/0294891 A1
(43) Pub. Date: Oct. 1, 2026

(54) METHODS OF TREATING ANHEDONIA

(71) Applicant: Neumora Therapeutics, Inc., Watertown, MA (US)

(72) Inventors: Daljit Singh Aurora, Thousand Oaks, CA (US); Andrew Ellis Jaffe, Baltimore, MD (US)

(21) Appl. No.: 19/474,389

(22) PCT Filed: Apr. 12, 2024
(86) PCT No.: PCT/US2024/024292

§ 371 Date: Oct. 10, 2025

Related U.S. Application Data
(60) Provisional application No. 63/459,240, filed on Apr. 13, 2023.

(51) Int. Cl. A61K 31/4709

(52) U.S. Cl. CPC A61K 31/4709

(57) ABSTRACT

The present disclosure relates methods of treating psychiatric disorders such as anhedonia with a kappa opioid receptor antagonist.

### CLAIM OF PRIORITY

This application claims the benefit of U.S. Provisional Application Ser. No. 63/459,240, filed on Apr. 13, 2023. The entire contents of which are hereby incorporated by reference in their entirety.

### FIELD

The present disclosure relates to methods for treating psychiatric disorders such as anhedonia associated with depression.

### BACKGROUND

Anhedonia—the loss of pleasure or lack of reactivity to pleasurable stimuli—is often associated with patients suffering from depression, and it remains a significant treatment challenge. In subjects with major depressive disorder (MDD), for example, anhedonia has been linked to poor outcomes, such as a reduced response to psychological and psychiatric interventions, and an increased suicide risk. See, e.g., Pizzagalli, Am. J. Psychiatry, Vol. 179, No. 7, pp. 458-469 (2022).

While antidepressant medications and cognitive behavioral therapy can be effective for some individuals, up to 20% do not respond to these interventions, and many of those who do respond, eventually relapse. Similarly, an estimated 50% of depressed individuals are only partially (inadequately) treated by available clinical interventions. See Al Harbi, Patient Prefer. Adherence, Vol. 6, pp. 369-388 (2012) and Gorwood, Dialogues Clin. Neurocsci., Vol. 10, No. 3, pp. 291-299 (2008). In the NIMH Sequenced Treatment Alternatives to Relieve Depression (STAR*D) study, approximately half of patients treated with a first-line antidepressant therapy had reduction of symptoms to at least half of the original intensity and only approximately one-third of patients achieved remission (Chan 2013). While these patients may eventually recover, many require a trial and error approach to therapy, and many will ultimately develop treatment resistant depression (TRD) over time, i.e., failure to adequately respond to two or more courses of antidepressant treatment, and/or may still exhibit lingering anhedonia. See, e.g., Sackheim, J. Clin. Psychiatry, Vol. 62, Suppl. 16, pp. 10-17 (2001) and Fava M., Biol. Psychiatry 53:649-59 (2003) and McIntyre, et al., J. Affect. Disord. 156:1-7 (2014). This can lead to even more serious conditions such as suicidal ideation and suicidality.

The lack of efficacy and the significant adverse events associated with the use of current antidepressants leads to high levels of treatment discontinuation (Zajecka 2000). Even with multiple consecutive treatments, only a small proportion of patients remain asymptomatic (Rush 2007). In the meantime, individuals continue to suffer from anhedonia, have a risk of self-harm, and experience a negative impact in their personal and professional lives. See, e.g., Burcusa and Iacono, Clin. Psychol. Rev. Vol. 27, No. 8, pp. 959-985 (2007). Thus, methods to treat anhedonia would have a substantial impact on public health.

### SUMMARY

The details of one or more embodiments are set forth in the description below. Other features, objects, and advantages of the invention will be apparent from the description and from the claims.

Some embodiments provide a method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject previously identified or diagnosed as having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having depression.

Some embodiments provide a method of treating anhedonia in a subject in need thereof, comprising:
(a) identifying a subject as having one or more symptoms of depression; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject previously identified or diagnosed as having depression, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof,
wherein the subject in need thereof has been previously identified or diagnosed as having depression.

Some embodiments provide a method of treating anhedonia in a subject in need thereof, consisting essentially of:
(a) identifying a subject as having one or more symptoms of depression; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a SHAPS score of from 31 to 56.

Some embodiments provide a method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a SHAPS score of from 37 to 56.

Some embodiments provide a method of treating anhedonia in a subject in need thereof, comprising:
(a) determining a first SHAPS score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(c) determining a second SHAPS score in the subject; wherein the second SHAPS score is less than the first SHAPS score.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject previously identified or diagnosed as having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof,
wherein the subject in need thereof has been previously identified or diagnosed as having depression.

Some embodiments provide a method of reducing the severity of anhedonia in a subject in need thereof, comprising:
(a) identifying a subject as having one or more symptoms of depression; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject previously identified or diagnosed as having depression, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having depression.

Some embodiments provide a method of reducing the severity of anhedonia in a subject in need thereof, consisting essentially of:
(a) identifying a subject as having one or more symptoms of depression; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a SHAPS score of from 31 to 56.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a SHAPS score of from 37 to 56.

Some embodiments provide a method of reducing the severity of anhedonia in a subject in need thereof, comprising:
(a) determining a first SHAPS score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(c) determining a second SHAPS score in the subject; wherein the second SHAPS score is less than the first SHAPS score.

Some embodiments provide a method of reducing the SHAPS score in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the SHAPS score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the SHAPS score in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

### DESCRIPTION OF THE FIGURES

The following drawings illustrate certain embodiments of the features and advantages of this disclosure. These embodiments are not intended to limit the scope of the appended claims in any manner. Like reference symbols in the drawings indicate like elements.

FIG. 1 illustrates the sensitivity analysis by last observation carried forward (LOCF) method (final efficacy population) for the clinical trial described in Example 1.

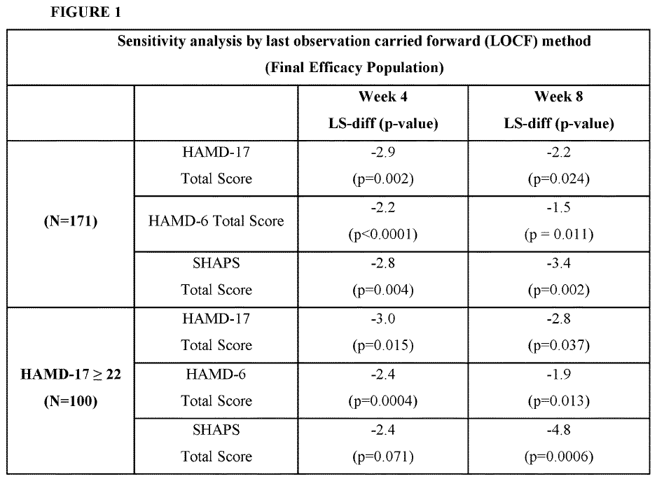

### DETAILED DESCRIPTION

### Definitions

To facilitate understanding of the disclosure set forth herein, a number of additional terms are defined below. Generally, the nomenclature used herein and the laboratory and clinical procedures in organic chemistry, medicinal chemistry, pharmacology, and psychiatry described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The compound 1-(6-ethyl-8-fluoro-4-methyl-3-(3-methyl-1,2,4-oxadiazol-5-yl)quinolin-2-yl)-N-(tetrahydro-2H-pyran-4-yl)piperidin-4 amine, or a pharmaceutically acceptable salt thereof, is a kappa opioid receptor antagonist having the structure.

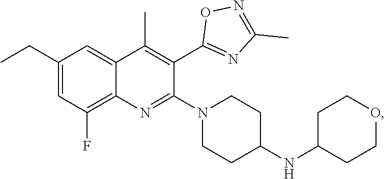 [SMILES: CCc1cc(F)c2nc(N3CCC(NC4CCOCC4)CC3)c(-c3nc(C)no3)c(C)c2c1]

or a pharmaceutically acceptable salt thereof described in, for example, U.S. Pat. No. 9,682,966.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

As used herein, the “subject” refers to a human. In some embodiments, the subject has experienced and/or exhibited at least one symptom of the disorder to be treated. “Treatment” or “therapy” of a subject refers to any type of intervention or process performed on, or the administration of an active agent to, the subject with the objective of reversing, alleviating, ameliorating, inhibiting, or slowing down, the onset, progression, development, severity, or recurrence of a symptom, complication, condition, or biochemical indicia associated with a disease, in whole or in part. In some embodiments, “treatment” includes resolution of a particular disorder, including a reduction in one or more symptoms of the disorder and/or a reduction in in the severity of one or more symptoms associated with the disorder.

Unless indicated otherwise, a “score” and a “total score” are used interchangeably herein, for example, HAMD-6 “Total Score” and HAMD-6 “Score.”

“Administering” or “administration” refer to the physical introduction of a therapeutic agent to a subject, using any of the various methods and delivery systems known to those skilled in the art. Routes of administration can include, for example, oral or intravenous administration. Administration can also be performed, for example, once, a plurality of times, and/or over one or more extended periods.

A “therapeutically effective amount” of a therapeutic agent is any amount of the agent that, when used alone or in combination with one or more additional therapies, slows down the onset of anhedonia or promotes regression of anhedonia evidenced by a decrease in severity of disorder symptoms, an increase in frequency and duration of symptom-free periods, or a ameliorating an impairment or disability due to the anhedonia (i.e., an amount sufficient to treat (as defined herein) the disorder). For example, a “therapeutically effective amount” of a therapeutic agent may result in amelioration, reduction, or elimination of at least one of the following symptoms: persistent sadness or anxiety, feelings of emptiness, hopelessness, pessimism, guilt, worthlessness, helplessness, a loss of interest or pleasure in hobbies and activities that were once enjoyed, decreased energy, fatigue, difficulty concentrating, remembering, or making decisions, insomnia, early-morning awakening, oversleeping, appetite loss, weight loss, overeating, weight gain, restlessness, irritability, and persistent physical symptoms that do not respond to treatment, such as headaches, digestive disorders, and chronic pain.

As used herein, a measure of a treatment effect is “clinically meaningful” based on the practical importance of a treatment effect. For example, whether the treatment effect has a real genuine, palpable, and/or noticeable effect on the subject (e.g., a lack of clinically meaningful effect occurs when the difference in the subject is small enough that it may be considered similar, such as prior to and after administration of a treatment as provided herein). One skilled in the art would recognize whether a particular effect is “clinically meaningful.” For example, a subject having a baseline score indicating severe depression (using any of the scales described herein) and a post-treatment score indicating remission of the severe depression would be a clinically meaningful effect.

A subject that is “not responsive” refers to a subject that has been, or is currently being, treated with one or more therapies that are not providing a clinically meaningful change towards the desired outcome (e.g., subjects that are not responsive includes patients that are refractory to a particular treatment). For example, a subject may exhibit no measurable change in response to therapy. A non-responsive subject could also, for example, exhibit a positive change in a depression scale score, but the change is not clinically meaningful.

As used herein, the “response rate” refers to the percentage of subjects that exhibit a clinically meaningful response to treatment with a particular agent, or combination of agents.

As used herein, a psychiatric evaluation or side effect profile test score that is “substantially similar” or “substantially the same” as a reference score, corresponds to the same score, with a skilled artisan understanding that particular test scores may vary to a reasonable extent (such as ±10%) while still describing a given value, due to, for example, experimental error, routine subject-to-subject evaluation, and routine statistical analysis.

The essential features of a “major depressive episode” or “major depression” is a period of at least 2 weeks during which there is either a depressed mood or the loss of interest or pleasure in nearly all activities. The individual must also experience at least four additional symptoms drawn from a list that includes changes in appetite or weight, sleep, and psychomotor activity, decreased energy; feelings of worthlessness or guilt; difficulty thinking, concentrating, or making decisions; or recurrent thoughts of death or suicidal ideation, plans or attempts. To be considered a major depressive episode, a symptom must have clearly worsened compared with the person's pre-episode status. The symptoms must persist for most of the day, nearly every day, for at least 2 consecutive weeks. The episode must be accompanied by clinically significant distress or impairment in social, occupational or other important areas of functioning (Diagnostic and Statistical Manual of Mental Disorders 4th Ed. DSM-IV, Pub. American Psychiatric Association, Washington, D.C.; p. 320, 327, 344-345). A “major depressive disorder” generally refers to a single or recurrent Major Depressive Episode which is not better accounted for by Schizophrenia, Delusional Disorder, or Psychotic Disorder Not Otherwise Specified, and also there has never been a Manic Episode, a Mixed Episode or a Hypomanic Episode (Diagnostic and Statistical Manual of Mental Disorders 4th Ed. DSM-IV, Pub. American Psychiatric Association, Washington, D.C.; pp. 344-345). The diagnosis of depression is generally based on evaluation by a qualified physician, generally a psychiatrist or by a psychologist. A “minor depressive disorder”, also referred to as “dysthymia”, has the characteristics of a major depressive disorder but presents itself without the intensity or severity of the symptoms associated with a “major depressive disorder”. “Late Life Major Depression”, referred to as “LLMD” or “late-onset depression” refers to depression, for example, the major and minor depressive disorders and depressive episodes described above, that occurs in a subject at about 60 years of age or older. The “risk factors” for depression include female gender, unmarried status, having stressful life events and lack of a social support network. Major depressive disorder is characterized by any of a number of symptoms, including persistent sadness or anxiety, or feelings of emptiness, hopelessness, pessimism, guilt, worthlessness, or helplessness.

As used in the methods described herein, the term “reducing” refers to a reduction in the indicated parameter relative to the baseline measurement (or measurements) of the same parameter in the subject taken prior to the initiation of administration or a kappa opioid receptor antagonist, or a reduction in the indicated parameter relative to the baseline measurement (or measurements) of the same parameter. In some embodiments, the same parameter is measured in a healthy subject (for example, a subject that does not have a psychiatric disorder as described herein). In some embodiments, the same parameter is measured relative to another treatment modality (for example, the standard of care treatment for a depression as described herein).

Similarly, the term “increasing,” as used herein, refers to an increase in the indicated parameter relative to the baseline measurement (or measurements) of the same parameter in the subject taken prior to the initiation of administration of a kappa opioid receptor antagonist, or an increase in the indicated parameter relative to the baseline measurement (or measurements) of the same parameter. In some embodiments, the same parameter is measured in a healthy subject (for example, a subject that does not have depression as described herein). In some embodiments, the same parameter is measured relative to another treatment modality (for example, the standard of care treatment for depression as described herein).

Measurements of certain parameters described herein can be qualitative (e.g., based on patient description of feelings) and/or quantitative (e.g., based on scale scores, as described herein). Subject feelings and/or behavior can be self-reported or assessed by a third party such as a family member, friend, physician, counselor, or other caregiver.

As described herein, any concentration range, percentage range, ratio range, or integer range is to be understood to include the value of any integer within the recited range and, when appropriate, fractions thereof (such as one tenth and one hundredth of an integer), unless otherwise indicated.

### INTRODUCTION

The presence of anhedonia is associated with difficulty in treating major depressive disorder (MDD). Research findings indicate that available therapies do not target depression-related motivational and reward-processing deficits sufficiently (APA 2000; Dunlop and Nemeroff 2007; McCabe et al., 2010; Nutt et al., 2007; Price et al., 2009; Shelton and Tomarken 2001) and that anhedonic symptoms predict inadequate treatment response (Spijker et al., 2001).

The presence of anhedonia is also associated with inadequate treatment response to antidepressant drugs (McMakin et al., 2012; Uher et al., 2012) and potentially also to psychological treatments (Craske et al., 2016). Unfortunately, research findings indicate that available therapies such as SSRIs do not target depression-related motivational and reward-processing deficits sufficiently (APA 2000; Dunlop and Nemeroff 2007; McCabe et al., 2010; Nutt et al., 2007; Price et al., 2009; Shelton and Tomarken 2001). Indeed, a significant number of subjects with depression do not achieve a sustained and complete response even after multiple therapeutic trials. See, e.g., Rush, et al., Psychol. Med. Vol. 52, pp. 419-432 (2022), which is incorporated by reference in its entirety.

The kappa opioid receptor (KOR) is a seven transmembrane-spanning G protein-coupled receptor encoded by the opioid receptor kappa 1 (OPRK1) gene. It functions as a receptor for endogenous ligands, as well as a receptor for various synthetic opioids. KOR plays a role in the perception of pain and mediating the hypolocomotor, analgesic and aversive actions of synthetic opioids. See e.g., Lalanne et al., Front Psychiatry (2014) 5:170.

Activation of kappa opioid receptors (KORs) produces negative affect. For example, KOR agonists produce dysphoric effects and elicit psychotomimetic properties in humans, as well as elicit place aversion and depressive-like affective behaviors in rodents. See Chavkin and Koob, (2016) Neuropsychopharmacology 41:373-374. One mechanism implicated in κ-mediated aversion is the modulation of mesolimbic dopamine circuitry, where KORs are expressed on dopamine terminals. Activation of KORs following systemic agonist treatment reduces dopamine release. See Chefer et al., Neuropsychopharmacology (2013) 38:2623-2631. Ablation of KORs from dopamine neurons KORs on BLA glutamatergic neurons that project to the medial PFC results in an anxiolytic phenotype, suggesting that these circuits are critical to the expression of negative affective-like behavior.

The present application is based, in part, on the surprising discovery that while certain kappa opioid receptor antagonists are unable to exert a clinically meaningful impact on anhedonia (e.g., anhedonia in subjects with depression, such as MDD), administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof provides a clinically meaningful reduction in symptoms of anhedonia and a concomitant increase in quality of life for those subjects. See, e.g., U.S. Pat. No. 11,266,627 (col. 38-41), and clinical trial results for 2019-000695-41 available on the European Union Clinical Trials Register (www.clinicaltrialsregister.eu/ctr-search/trial/2019-000695-41/results), which are hereby incorporated by reference in their entirety for the limited purpose of comparative data.

### Methods of Treatment

Some embodiments provide a method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject previously identified or diagnosed as having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having depression.

Some embodiments provide a method of treating anhedonia in a subject in need thereof, comprising:
(a) identifying a subject as having one or more symptoms of depression; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject previously identified or diagnosed as having depression, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof,
wherein the subject in need thereof has been previously identified or diagnosed as having depression.

Some embodiments provide a method of treating anhedonia in a subject in need thereof, consisting essentially of:
(a) identifying a subject as having one or more symptoms of depression; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject previously identified or diagnosed as having depression, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof,
wherein the subject in need thereof has been previously identified or diagnosed as having depression.

Some embodiments provide a method of reducing the severity of anhedonia in a subject in need thereof, comprising:
(a) identifying a subject as having one or more symptoms of depression; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject previously identified or diagnosed as having depression, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, consisting essentially of: administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject in need thereof has been previously identified or diagnosed as having depression.

Some embodiments provide a method of reducing the severity of anhedonia in a subject in need thereof, consisting essentially of:
(a) identifying a subject as having one or more symptoms of depression; and
(b) administering a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a SHAPS score of from 31 to 56.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a Snaith-Hamilton Pleasure Scale (SHAPS) score of from 37 to 56.

Some embodiments provide a method of reducing the severity of anhedonia in a subject in need thereof, comprising:
(a) determining a first SHAPS score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(c) determining a second SHAPS score in the subject; wherein the second SHAPS score is less than the first SHAPS score.

Some embodiments provide a method of reducing the SHAPS score in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the SHAPS score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the SHAPS score in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the HAMD-17 Score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HAMD-17 Total Score of 22 to 52. For example, some embodiments provide a method of reducing the HAMD-17 Score in a subject having anhedonia by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a Hamilton Depression Rating Scale 17 (HAMD-17) Total Score of 27 to 52, 32 to 52, 37 to 52, 42 to 52, or 47 to 52.

Some embodiments provide a method of reducing the HAMD-17 Total Score in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the HAMD-17 Total Score in a subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-17 Total Score of the subject is decreased by about 2 to about 4 (e.g., by about 3) after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. Additionally, or in the alternative, some embodiments provide a method of reducing the HAMD-17 Total Score in a subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-17 Total Score of the subject is decreased by about 2 to about 4 (e.g., by about 3) after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the HAMD-17 Total Score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the HAMD-17 Total Score in a subject having anhedonia, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-17 Total Score of the subject is decreased by about 2 to about 4 (e.g., by about 3) after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. Additionally, or in the alternative, some embodiments provide a method of reducing the HAMD-17 Total Score in a subject having anhedonia, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-17 Total Score of the subject is decreased by about 2 to about 4 (e.g., by about 3) after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the HAMD-17 Total Score in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the HAMD-17 Total Score in a subject previously identified or diagnosed as having anhedonia, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-17 Total Score of the subject is decreased by about 2 to about 4 (e.g., by about 3) after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. Additionally, or in the alternative, some embodiments provide a method of reducing the HAMD-17 Total Score in a subject previously identified or diagnosed as having anhedonia, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-17 Total Score of the subject is decreased by about 2 to about 4 (e.g., by about 3) after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the HAMD-6 Total Score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HAMD-6 (abbreviated 6 item version derived from HAMD-17) Total Score of from 11 to 22. For example, some embodiments provide a method of treating (e.g., reducing the severity of) anhedonia in a subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HAMD-6 Total Score of from 12 to 22, from 14 to 22, from 16 to 22, from 18 to 22, or from 20 to 22.

Some embodiments provide a method of reducing the HAMD-6 Total Score in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the HAMD-6 Total Score in a subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-6 Total Score of the subject is decreased by about 1 to about 3 (e.g., by about 2) after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. Additionally, or in the alternative, some embodiments provide a method of reducing the HAMD-6 Total Score in a subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-6 Total Score of the subject is decreased by about 1 to about 3 (e.g., by about 2) after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the HAMD-6 Total Score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the HAMD-6 Total Score in a subject having anhedonia, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-6 Total Score of the subject is decreased by about 1 to about 3 (e.g., by about 2) after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. Additionally, or in the alternative, some embodiments provide a method of reducing the HAMD-6 Total Score in a subject having anhedonia, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-6 Total Score of the subject is decreased by about 1 to about 3 (e.g., by about 2) after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the HAMD-6 Total Score in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. For example, some embodiments provide a method of reducing the HAMD-6 Total Score in a subject previously identified or diagnosed as having anhedonia, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-6 Total Score of the subject is decreased by about 1 to about 3 (e.g., by about 2) after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. Additionally, or in the alternative, some embodiments provide a method of reducing the HAMD-6 Total Score in a subject previously identified or diagnosed as having anhedonia, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the HAMD-6 Total Score of the subject is decreased by about 1 to about 3 (e.g., by about 2) after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the Hospital Anxiety and Depression Scale (HADS) score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a Hospital Anxiety and Depression Scale (HADS) score of 8 to 10.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HADS score of 11 to 14.

Some embodiments provide a method of reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HADS score of 15 to 21. For example, some embodiments provide a method of treating (e.g., reducing the severity of) anhedonia in a subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HADS score of 17 to 21, or 19 to 21.

Some embodiments provide a method of reducing the HADS score in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the HADS score is reduced in the subject after four weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the HADS score is reduced in the subject after eight weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the HADS score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the HADS score is reduced in the subject after four weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the HADS score is reduced in the subject after eight weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the HADS score in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the HADS score is reduced in the subject after four weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the HADS score is reduced in the subject after eight weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the Hamilton Anxiety Rating Scale (HAM-A) score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a Hamilton Anxiety Rating Scale (HAM-A) score of 6 to 14. For example, some embodiments provide a method of treating (e.g., reducing the severity of) anhedonia in a subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HAM-A score of 8 to 14, 10 to 14, or 12 to 14.

Some embodiments provide a method reducing the severity of anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HAM-A score of 15 to 28. For example, some embodiments provide a method of treating (e.g., reducing the severity of) anhedonia in a subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HAM-A score of 16 to 28, 18 to 28, 20 to 28, 22 to 28, 24 to 28, or 26 to 28.

Some embodiments provide a method of reducing the HAM-A score in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HAM-A score of 29 to 52. For example, some embodiments provide a method of treating (e.g., reducing the severity of) anhedonia in a subject in need thereof, by administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a HAM-A score of 30 to 52, 32 to 52, 34 to 52, 36 to 52, 38 to 52, 40 to 52, 42 to 52, 44 to 52, 46 to 52, 48 to 52, or 50 to 52.

Some embodiments provide a method of reducing the HAM-A score in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the HAM-A score is reduced in the subject after four weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the HAM-A score is reduced in the subject after eight weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the HAM-A score in a subject having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the HAM-A score is reduced in the subject after four weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the HAM-A score is reduced in the subject after eight weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of reducing the HAM-A score in a subject previously identified or diagnosed as having anhedonia, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the HAM-A score is reduced in the subject after four weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the HAM-A score is reduced in the subject after eight weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of treating anhedonia in a subject in need thereof, comprising: (a) determining two or more of a CGI-S score, a HAMD-17 score, a SHAPS score, a HAM-A score, a STAI subscale score, a HADS score (e.g., total and subscale), a PHQ-9 score (e.g., total PHQ-9 score), and a SDS score (e.g., SDS total score) in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, comprising: (a) determining two or more of a CGI-S score, a HAMD-17 score, a SHAPS score, a HAM-A score, a STAI subscale score, a HADS score (e.g., total and subscale), a PHQ-9 score (e.g., total PHQ-9 score), and a SDS score (e.g., SDS total score) in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, comprising: (a) determining two or more of a CGI-S score, a HAMD-17 score, a SHAPS score, a HAM-A score, a STAI subscale score, a HADS score (e.g., total and subscale), a PHQ-9 score (e.g., total PHQ-9 score), and a SDS score (e.g., SDS total score) in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, comprising: (a) determining two or more of a CGI-S score, a HAMD-17 score, a SHAPS score, a HAM-A score, a STAI subscale score, a HADS score (e.g., total and subscale), a PHQ-9 score (e.g., total PHQ-9 score), and a SDS score (e.g., SDS total score) in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, comprising: (a) determining two or more of a first CGI-S score, a first HAMD-17 score, a first SHAPS score, a first HAM-A score, a first STAI subscale score, a first HADS score (e.g., total and subscale), a first PHQ-9 score (e.g., total PHQ-9 score), and a first SDS score (e.g., SDS total score) in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and (c) determining two or more of a second CGI-S score, a second HAMD-17 score, a second SHAPS score, a second HAM-A score, a second STAI subscale score, a second HADS score (total and subscale), a second PHQ-9 score (e.g., total PHQ-9 score), and a second SDS score (e.g., SDS total score) in the subject; wherein each of the second scores independently has a one or more point improvement relative to the two or more first scores. Some embodiments provide a method of reducing the severity of anhedonia in a subject in need thereof, comprising: (a) determining two or more of a first CGI-S score, a first HAMD-17 score, a first SHAPS score, a first HAM-A score, a first STAI subscale score, a first HADS score (e.g., total and subscale), a first PHQ-9 score (e.g., total PHQ-9 score), and a first SDS score (e.g., SDS total score) in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and (c) determining two or more of a second CGI-S score, a second HAMD-17 score, a second SHAPS score, a second HAM-A score, a second STAI subscale score, a second HADS score (total and subscale), a second PHQ-9 score (e.g., total PHQ-9 score), and a second SDS score (e.g., SDS total score) in the subject; wherein each of the second scores independently has a one or more point improvement relative to the two or more first scores. In some embodiments, the second CGI-S score is less than the first CGI-S score, the second HAMD-17 score is less than the first second HAMD-17 score, the second SHAPS score is less than the first SHAPS score, the second HAM-A score is less than the first HAM-A score, the second STAI subscale score is less than the first STAI subscale score, the second HADS score (e.g., total and subscale) is less than the first HADS score, the second PHQ-9 score (e.g., total PHQ-9 score) is less than the first PHQ-9 score, and/or the second SDS score (e.g., SDS total score) is less than the first SDS score after four weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the second CGI-S score is less than the first CGI-S score, the second HAMD-17 score is less than the first second HAMD-17 score, the second SHAPS score is less than the first SHAPS score, the second HAM-A score is less than the first HAM-A score, the second STAI subscale score is less than the first STAI subscale score, the second HADS score (e.g., total and subscale) is less than the first HADS score, the second PHQ-9 score (e.g., total PHQ-9 score) is less than the first PHQ-9 score, and/or the second SDS score (e.g., SDS total score) is less than the first SDS score after eight weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of treating anhedonia in a subject in need thereof, comprising: (a) determining two or more of a HAMD-17 score, a SHAPS score, and a STAI subscale score in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of reducing the severity of anhedonia in a subject in need thereof, comprising: (a) determining two or more of a HAMD-17 score, a SHAPS score, and a STAI subscale score in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, comprising: (a) determining two or more of a first HAMD-17 score, a first SHAPS score, and a first STAI subscale score in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and (c) determining one or more of a second HAMD-17 score, a second SHAPS score, and a second STAI subscale score in the subject; wherein each of the second scores independently has a one or more point improvement relative to the two or more first scores. Some embodiments provide a method of reducing the severity of anhedonia in a subject in need thereof, comprising: (a) determining two or more of a first HAMD-17 score, a first SHAPS score, and a first STAI subscale score in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and (c) determining one or more of a second HAMD-17 score, a second SHAPS score, and a second STAI subscale score in the subject; wherein each of the second scores independently has a one or more point improvement relative to the two or more first scores. In some embodiments, the second HAMD-17 score is less than the first second HAMD-17 score, the second SHAPS score is less than the first SHAPS score, and/or the second STAI subscale score is less than the first STAI subscale score after four weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the second HAMD-17 score is less than the first second HAMD-17 score, the second SHAPS score is less than the first SHAPS score, and/or the second STAI subscale score is less than the first STAI subscale score after eight weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

Some embodiments provide a method of treating anhedonia in a subject in need thereof, comprising: (a) determining one or more of a SHAPS score, a HAM-A score, a HADS score (e.g., total and subscale), and a SDS score (e.g., SDS total score) in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. Some embodiments provide a method of reducing the severity of anhedonia in a subject in need thereof, comprising: (a) determining one or more of a SHAPS score, a HAM-A score, a HADS score (e.g., total and subscale), and a SDS score (e.g., SDS total score) in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. Some embodiments provide a method of treating anhedonia in a subject in need thereof, comprising: (a) determining one or more of a first SHAPS score, a first HAM-A score, a first HADS score (e.g., total and subscale), and a first SDS score (e.g., SDS total score) in the subject; (b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and (c) determining one or more of a second SHAPS score, a second HAM-A score, a second HADS score (total and subscale), a second SDS score (e.g., SDS total score), and a CGI-I score in the subject; wherein each of the second scores independently has a one or more point improvement relative to the two or more first scores. In some embodiments, the second SHAPS score is less than the first SHAPS score, the second HAM-A score is less than the first HAM-A score, the second HADS score (e.g., total and subscale) is less than the first HADS score, the second SDS score (e.g., SDS total score) is less than the first SDS score, and/or the CGI-I score is ≤2 after four weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof. In some embodiments, the second SHAPS score is less than the first SHAPS score, the second HAM-A score is less than the first HAM-A score, the second HADS score (e.g., total and subscale) is less than the first HADS score, the second SDS score (e.g., SDS total score) is less than the first SDS score, and/or the CGI-I score is ≤2 after eight weeks of once daily administration of the 1-[6-ethyl-8-fluoro-4-methyl-3-(3-methyl-1,2,4-oxadiazol-5-yl)quinolin-2-yl]-N-(oxan-4-yl)piperidin-4-amine, or the pharmaceutically acceptable salt thereof.

In some embodiments, the depression is major depressive disorder (MDD) or treatment resistant depression (TRD).

In some embodiments, the depression is major depressive disorder (MDD).

In some embodiments, the depression is treatment resistant depression (TRD). Treatment resistant depression, as described herein, includes depression that does not adequately respond to a course of treatment for depression within a defined time period (e.g., as defined by a clinician). An inadequate response includes, but is not limited to, less than an 80% reduction in depressive symptoms, less than a 75% reduction in depressive symptoms, less than a 70% reduction in depressive symptoms, less than a 65% reduction in depressive symptoms, less than a 60% reduction in depressive symptoms, less than a 55% reduction in depressive symptoms, less than a 50% reduction in depressive symptoms, less than a 45% reduction in depressive symptoms, less than a 40% reduction in depressive symptoms, less than a 35% reduction in depressive symptoms, or less than a 30% reduction in depressive symptoms. In some embodiments, the subject fails to adequately respond to two or more courses of antidepressant treatment, such as two, three, four, of five courses of treatment.

In some embodiments, the depression of a subject demonstrates a partial response, inadequate response, or residual symptomatology following treatment. In some embodiments, the depression is difficult-to-treat depression (DTD). Potential parameters that can be used, for example, to define whether a subject has DTD and/or characterize a subject within a sub-group of DTD include, but are not limited to early life trauma, concurrent medications, functional impairment, family history, variability of symptoms, adherence to treatment protocols, symptom features (such as anhedonia), the course of depression, cormorbid psychiatric conditions or other general medical conditions, the number and sequence of failed treatments, and the types of failed treatment (and types of failure, such as non-responsiveness or non-compliance due to side effects). Subjects with DTD may be non-responsive to current antidepressant therapies, or may exhibit an initial response that wanes over time, ultimately with the depressive symptoms returning despite continuing treatment. Accordingly, in some embodiments, the methods of treating DTD described herein further comprise additional clinical evaluation of (i) the durability of benefit of treatment, (ii) the side effect burden of treatment, if any, and/or (iii) presence of a sustained impact on quality of life and/or or daily function. Such additional clinical evaluations can occur before and/or during treatment, and can be conducted by a clinician and/or self-reported by the subject.

In some embodiments, the depression further comprises anxiety.

In some embodiments, the subject maintains about the same weight before administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof and after administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time. In some embodiments, this period of time is, for example, about 1 month, about 2 months, about 3 months, about 4, months, about 5 months, about 6 months, about 7 months, about 8 months, about 9 months, about 10 months, about 11 months, about 1 year, about 2 years, about 3 years, about 4, years, about 5 years, about 6 years, about 7 years, about 8 years, about 9 years, or about 10 years or more.

In some embodiments, the subject maintains about the level of sexual function before administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof and after administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time. In some embodiments, this period of time is, for example, about 1 month, about 2 months, about 3 months, about 4, months, about 5 months, about 6 months, about 7 months, about 8 months, about 9 months, about 10 months, about 11 months, about 1 year, about 2 years, about 3 years, about 4, years, about 5 years, about 6 years, about 7 years, about 8 years, about 9 years, or about 10 years or more. The level of sexual function can be assessed, for example, by clinical evaluation, self-reporting, and/or reporting from the subject's partner(s).

In some embodiments, the subject does not experience significant weight gain during treatment with 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. Significant weight gain refers to a 10% or higher increase in body weight.

In some embodiments, the subject does not experience sexual dysfunction during treatment with 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the method comprises a reduction in one or more depressive symptoms in the subject. Depressive symptoms include, but are not limited to dysthymia, feelings of sadness, tearfulness, emptiness or hopelessness; angry outbursts, irritability and/or frustration, even over small matters; loss of interest or pleasure in most or all normal activities; sleep disturbances; insomnia; sleeping too much; tiredness and/or lack of energy, reduced appetite and/or weight loss; increased cravings for food; weight gain; anxiety; agitation; restlessness; slowed thinking; slowed speaking; slowed body movements; feelings of worthlessness; feelings of guilt; fixating on past failures; self-blame; trouble thinking, concentrating, making decisions, and/or remembering things; frequent and/or recurrent thoughts of death, suicidal thoughts, suicide attempts, or suicide; and unexplained physical problems, such as back pain and/or other chronic pain, digestive disorders, and/or headaches.

In some embodiments, the subject has a SHAPS score of from 31 to 56 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the subject has a SHAPS score of from 37 to 56 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the subject has a SHAPS score of from 31 to 37 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, the subject has a SHAPS score of from 31 to 56, 31 to 50, 31 to 44, 31 to 37, 37 to 56, 43 to 56, or 50 to 56.

Some embodiments provide a method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a SHAPS score of from 31 to 56.

Some embodiments provide a method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a SHAPS score of from 37 to 56.

Some embodiments provide a method of treating anhedonia in a subject in need thereof, consisting essentially of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a SHAPS score of from 31 to 56.

Some embodiments provide a method of treating anhedonia in a subject in need thereof, consisting essentially of administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof, wherein the subject has been previously identified as having a SHAPS score of from 37 to 56.

In some embodiments, the subject has been previously identified as having a SHAPS score of from 31 to 56, 31 to 50, 31 to 44, 31 to 37, 37 to 56, 43 to 56, or 50 to 56. In some embodiments, the subject has been previously identified as having a SHAPS score of from 31 to 37.

In some embodiments, the SHAPS score of the subject is decreased by about 2 to about 4 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is 29 or less, or 27 or less, after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is 35 or less, or 33 or less, after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is decreased by about 3 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is from 27 to 29 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is from 33 to 35 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is decreased by about 4 to about 6 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is 27 or less, or 25 or less, after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is 33 or less, or 31 or less, after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is decreased by about 5 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is from 25 to 27 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the SHAPS score of the subject is from 31 to 33 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

Some embodiments provide a method of treating anhedonia in a subject in need thereof, comprising:
(a) determining a first SHAPS score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(c) determining a second SHAPS score in the subject; wherein the second SHAPS score is less than the first SHAPS score.

Some embodiments provide a method of treating anhedonia in a subject in need thereof, consisting essentially of:
(a) determining a first SHAPS score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(c) determining a second SHAPS score in the subject; wherein the second SHAPS score is less than the first SHAPS score.

Some embodiments provide a method of treating anhedonia in a subject in need thereof, comprising:
(a) determining a first SHAPS score in the subject;
(b) determining a first HAMD-17 Total Score in the subject;
(c) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(d) determining a second SHAPS score in the subject;
(e) determining a second HAMD-17 Total Score in the subject;
wherein the second SHAPS score is less than the first SHAPS score and/or the second HAMD-17 Total Score is less than the first HAMD-17 Total Score.

Some embodiments provide a method of treating anhedonia in a subject in need thereof, consisting essentially of:
(a) determining a first SHAPS score in the subject;
(b) determining a first HAMD-17 Total Score in the subject;
(c) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof for a period of time; and
(d) determining a second SHAPS score in the subject;
(e) determining a second HAMD-17 Total Score in the subject;
wherein the second SHAPS score is less than the first SHAPS score and/or the second HAMD-17 Total Score is less than the first HAMD-17 Total Score.

In some embodiments, the period of time is about 1 week to about 52 weeks. In some embodiments, the period of time is about 1 week to about 6 weeks, about 4 weeks to about 8 weeks, about 6 weeks to about 12 weeks, about 10 weeks to about 16 weeks, about 12 weeks to about 24 weeks, about 16 weeks to about 30 weeks, about 24 weeks to about 36 weeks, about 30 weeks to about 40 weeks, about 36 weeks to about 44 weeks, or about 40 weeks to about 52 weeks. In some embodiments, the period of time is about 4 weeks to about 26 weeks. In some embodiments, the period of time is about 4 weeks to about 12 weeks. In some embodiments, the period of time is about 4 weeks to about 8 weeks.

In some embodiments, the first SHAPS score is from 31 to 56, 31 to 50, 31 to 44, 31 to 37, 37 to 56, 43 to 56, or 50 to 56. In some embodiments, the first SHAPS score is from 31 to 37. In some embodiments, the first SHAPS score is from 31 to 56. In some embodiments, the first SHAPS score is from 37 to 56.

In some embodiments, the first HAMD-17 Total Score is from 19 to 52. In some embodiments, the second HAMD-17 Total Score is lower than the first HAMD-17 Total Score by 2 to 8.

In some embodiments, the second SHAPS score is lower than the first SHAPS score by about 2 to about 5. In some embodiments, the second SHAPS score is lower than the first SHAPS score by 2 to 5. In some embodiments, the second SHAPS score is from 29 to 50. In some embodiments, the second SHAPS score is from 26 to 47. In some embodiments, the second SHAPS score is from 35 to 50. In some embodiments, the second SHAPS score is from 32 to 47. In some embodiments, the second SHAPS score is less than 29.

In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is administered once daily for the period of time, as described herein.

In some embodiments, the subject has previously been administered one or more antidepressant medications. In some embodiments, the subject has previously been administered one, two, or three antidepressant medications. In some embodiments, the subject has previously been administered one or two antidepressant medications. In some embodiments, the subject has previously been administered one antidepressant medication.

In some embodiments, the subject did not exhibit a clinically meaningful response to the one or more previously administered antidepressant medications.

In some embodiments, the one or more previously administered antidepressant medications are selected from selective serotonin reuptake inhibitors, selective norepinephrine reuptake inhibitors, and kappa opioid receptor antagonists.

In some embodiments, the one or more previously administered antidepressant medications are selected from selective serotonin reuptake inhibitors and selective norepinephrine reuptake inhibitors.

In some embodiments, the subject has been previously administered one or more selective serotonin reuptake inhibitors.

In some embodiments, the subject has been previously administered one or more kappa opioid receptor antagonists. In some embodiments, the kappa opioid receptor antagonist is aticaprant (4-(4-(((2S)-2-(3,5-dimethylphenyl)-1-pyrrolidinyl)methyl)phenoxy)-3-fluorobenzamide).

Some embodiments provide a method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof;
wherein the subject was previously administered aticaprant and discontinued treatment with aticaprant.

Some embodiments provide a method of treating anhedonia in a subject previously administered aticaprant, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject did not exhibit a clinically meaningful response after administration of aticaprant for a period of time.

In some embodiments, the subject was previously administered about 5 mg to about 20 mg of aticaprant. In some embodiments, the subject was previously administered about 10 mg of aticaprant.

In some embodiments, the subject has not been previously administered one or more antidepressant medications. In some embodiments, the subject has not been previously administered a selective serotonin reuptake inhibitor. In some embodiments, the subject has not been previously administered a selective norepinephrine reuptake inhibitor. In some embodiments, the subject has not been previously administered a kappa opioid receptor antagonist.

In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is from about 20 mg/day to about 160 mg/day. For example, about 20 mg/day to about 80 mg/day, about 40 mg/day to about 100 mg/day, about 60 mg/day to about 120 mg/day, about 80 mg/day to about 140 mg/day, or about 100 mg/day to about 160 mg/day.

In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is from about 20 mg/day to about 80 mg/day. In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is from about 80 mg/day to about 120 mg/day. In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is from about 120 mg/day to about 160 mg/day.

In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is 40 mg/day. In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is 80 mg/day. In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is 120 mg/day. In some embodiments, the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is 160 mg/day.

In some embodiments, the 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is administered orally. In some embodiments, the 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof is administered once per day.

In some embodiments, the subject has not had a score of “YES” on C-SSRS Item 4 or Item 5 within 3 months prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has not had a score of “YES” on C-SSRS Item 4 within 3 months prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has not had a score of “YES” on C-SSRS Item 5 within 3 months prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the subject has a HAMD-6 Total Score of from 11 to 22 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the HAMD-6 Total Score of the subject is decreased by about 1 to about 3 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the HAMD-6 Total Score of the subject is decreased by about 1 to about 3 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the subject has a HAMD-17 Total Score of 19 to 52 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the subject has a HAMD-17 Total Score of 22 to 52 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the HAMD-17 Total Score of the subject is decreased by about 2 to about 4 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof. In some embodiments, the HAMD-17 Total Score of the subject is decreased by about 2 to about 4 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine, or a pharmaceutically acceptable salt thereof.

In some embodiments, the 1-[6-ethyl-8-fluoro-4-methyl-3-(3-methyl-1,2,4-oxadiazol-5-yl)quinolin-2-yl]-N-(oxan-4-yl)piperidin-4-amine is administered as the free base.

In some embodiments, the 1-[6-ethyl-8-fluoro-4-methyl-3-(3-methyl-1,2,4-oxadiazol-5-yl)quinolin-2-yl]-N-(oxan-4-yl)piperidin-4-amine is administered as a pharmaceutically acceptable salt.

In some embodiments, the subject is a human.

Measuring Depression and/or Anhedonia

Many methods can be used to measure anhedonia and/or depression in a subject. Non-limiting examples are described herein. When a scale score or change of score is described, for example, the scales discussed below, it can be the score or change of score for a particular subject (e.g., such a score would typically be an integer) or an average of the score of two or more subjects (e.g., a score that could be an integer or between integers).

### Hamilton Depression Rating Scale 17

The Hamilton Depression Rating Scale 17 (also referred to as HAMD, HRSD, HDRS, or HAMD-17) is a 17-item version of a 21-item semi-structured clinician-administered interview scale used to assess severity of, and change in, depressive symptoms in an adult patient diagnosed as suffering from depression. See, e.g., Hamilton, J. Neurol. Neurosurg. Psychiatry. 1960 Feb. 23:56-62 (1960); and Williams, Eur. Arch. Psychiatry. Clin. Neurosci. 251 (suppl 2):116 (2001).

The 17-item version focuses more on somatic symptoms than on cognitive or affective symptoms, and includes: 1) depressed mood; 2) guilt; 3) suicide; 4) insomnia-initial; 5) insomnia-middle; 6) insomnia-delayed; 7) work and interests; 8) psychomotor retardation; 9) agitation; 10) psychic anxiety; 11) somatic anxiety; 12) gastrointestinal somatic symptoms; 13) general somatic symptoms; 14) genital somatic symptoms; 15) hypochondriasis; 16) insight; 17) loss of weight. Each item either is graded on a 5-point scale (0-4), with a score of (0) representing absent; (1), mild; (2, 3), moderate; or (4), severe symptoms; or, on a 3-point scale (0-2), with a score of (0) representing absent; (1), slight or doubtful; and (2), clearly present symptoms. The 17 items are added to provide a total score, with a maximum score of 52. A higher score is indicative of more severe symptoms, for example, HAM-D score level of depression of 0-7 is considered not depressed; 8-13 is considered subthreshold to mild; 14-18 is considered mild to moderate; 19-22 is considered moderate to severe; and >23 is considered severe to very severe.

The additional four items on the 21-item scale are diurnal variation, depersonalization/derealization, paranoid symptoms, and obsessional and compulsive symptoms. These criterion are generally not used as they may be less likely due to the disease, they were infrequent, and/or they are not considered markers of disease severity. See, e.g., Miller et al., Psych. Res. 14:131-142 (1984) and Hamilton, Br. J. Soc. Clin. Psychol. 6 (4): 278-96 (1967). There is also an abbreviated 6 item version (HAMD-6) derived from HAMD-17 and scoring the following 6 items from HAMD-17: item 1-depressed mood; item 2-guilt; item 7-work and interest; item 8-psychomotor retardation; item 10-psychic anxiety; and item 13-general somatic symptoms. The HAMD-6 Total Score can be up to 22, with a higher score indicating more severe symptoms.

### Clinical Global Impression of Improvement (CGI-I)

Clinical Global Impression of Improvement (CGI-I) score is a subscale of Clinical Global Impression (CGI), a retrospective assessment that is a measure of symptom severity, treatment response and the efficacy of treatments in treatment studies, completed by the treating physician at baseline and at subsequent clinic visits to document any change in target symptoms documented at baseline. Thus, CGI-I provides overall comparison of the patient's baseline condition with his current state.

Each time the patient is seen after medication has been initiated, the clinician compares the patient's overall clinical condition to the one week period just prior to the initiation of medication use (the so-called baseline visit), and the following one query only is rated on the 7-point scale: Compared to the patient's condition at admission to the project [prior to medication initiation], this patient's condition is: 1=very much improved since the initiation of treatment (nearly all better; good level of functioning; minimal symptoms; represents a very substantial change); 2=much improved (notably better with significant reduction of symptoms; increase in the level of functioning but some symptoms remain); 3=minimally improved (slightly better with little or no clinically meaningful reduction of symptoms. Represents very little change in basic clinical status, level of care, or functional capacity); 4=no change from baseline (the initiation of treatment) (symptoms remain essentially unchanged); 5=minimally worse (slightly worse but may not be clinically meaningful; may represent very little change in basic clinical status or functional capacity); 6=much worse (clinically significant increase in symptoms and diminished functioning); 7=very much worse since the initiation of treatment (severe exacerbation of symptoms and loss of functioning). See Busner and Targum, Psychiatry, 4(7), 28-37 (2007).

### Snaith-Hamilton Pleasure Scale (SHAPS)

The Snaith-Hamilton Pleasure Scale (SHAPS) score is a 14-item self-administered questionnaire used to measure hedonic capacity. The subjects indicate whether they experience pleasure in performing particular activities or experiences related to social interaction, food and drink, sensory experience, and interest/pastimes.

Specifically, the 14 items include: (1) I would enjoy my favorite television or radio program; (2) I would enjoy being with my family or close friends; (3) I would find pleasure in my hobbies and pastimes; (4) I would be able to enjoy my favorite meal; (5) I would enjoy a warm bath or refreshing shower; (6) I would find pleasure in the scent of flowers or the smell of a fresh sea breeze or freshly baked bread; (7) I would enjoy seeing other people's smiling faces; (8) I would enjoy looking smart when I have made an effort with my appearance; (9) I would enjoy reading a book, magazine or newspaper; (10) I would enjoy a cup of tea or coffee or my favorite drink; (11) I would find pleasure in small things, e.g. bright sunny day, a telephone call from a friend; (12) I would be able to enjoy a beautiful landscape or view; (13) I would get pleasure from helping others; (14) I would feel pleasure when I receive praise from other people.

Each of the items has four response categories: Definitely/Strongly Agree; Agree; Disagree; and Definitely/Strongly Disagree, which are scored as a 4, 3, 2, or 1, respectively. Thus, the SHAPS are scored as the sum of the 14 items so that total scores ranged from 14 to 56. A higher total SHAPS score indicates higher levels of current anhedonia. See, e.g., Snaith, et al., Br. J. Psychiatry, 167(1), 99-103 (1995) and Snaith, Psychol. Med. 23: 957-966 (1993).

### Hospital Anxiety and Depression Scale (HADS)

The Hospital Anxiety and Depression Scale (HADS) is a self-assessment mood scale that measures anxiety and depression. The HADS includes two conjoint 7-item subscales, one specifically targeted at anxiety (HADS-A) and one focusing on depression (HADS-D). HADS excludes many somatic symptoms for example dizziness and sleep disturbance although does include, for example, psychomotor agitation. The depression scale focuses on anhedonia. See, e.g., Zigmond and Snaith, Acta Psychiatrica Scandinavica, 67: 361-370 (1983) and Stem, Occupat. Med., 64(5): 393-394 (2014).

The 14 items include: (1) I feel tense or wound up; (2) I get a sort of frightened feeling as if something awful is about to happen; (3) Worrying thoughts go through my mind; (4) I can sit at ease and feel relaxed; (5) I get a sort of frightened feeling like ‘butterflies’ in the stomach; (6) I feel restless as I have to be on the move; (7) I get sudden feelings of panic; (8) I still enjoy the things I used to enjoy; (9) I can laugh and see the funny side of things; (10) I feel cheerful; (11) 1 feel as if I am slowed down; (12) I have lost interest in my appearance; (13) I look forward with enjoyment to things; (14) I can enjoy a good book or radio or TV program.

Each item is coded from 0 (no presence) to 3 (severe). The scores for anxiety and depression thus range from 0 to 21, with higher scores indicating more severe anxiety or depression. Scores of less than 7 indicate the subject is not anxious or depressed; Scores of 8-10 indicate mild anxiety or depression; 11-14=Scores of indicate moderate anxiety or depression; and Scores of 15-21 indicate severe anxiety or depression. See, e.g., Djukanovic, et al., Health Qual. Life Outcomes, 15(193) (2017) and Snaith, Health Qual. Life Outcomes, 1(29) (2003).

### Hamilton Anxiety Rating Scale (HAM-A) Score

The Hamilton Anxiety Rating Scale (HAM-A, HARS) score is a clinician-based questionnaire for measuring the severity of anxiety symptoms. The scale includes 14 items, each defined by a series of symptoms, and measures both psychic anxiety (mental agitation and psychological distress) and somatic anxiety (physical complaints related to anxiety). Specifically: (1) anxious mood; (2) tension (including startle response, fatigability, restlessness); (3) fears (including of the dark/strangers/crowds); (4) insomnia; (5) ‘intellectual’ (poor memory/difficulty concentrating); (6) depressed mood (including anhedonia); (7) somatic symptoms (including aches and pains, stiffness, bruxism); (8) sensory (including tinnitus, blurred vision); (9) cardiovascular (including tachycardia and palpitations); (10) respiratory (chest tightness, choking); (11) gastrointestinal (including irritable bowel syndrome-type symptoms); (12) genitourinary (including urinary frequency, loss of libido); (13) autonomic (including dry mouth, tension headache); and (14) observed behavior at interview (restless, fidgety, etc.). See, e.g., Hamilton, Br. J. Med. Psychol. 32:50-55 (1959) and Thompson, Occupat. Med., 65(7): 601, (2015).

Each item is scored from 0-4, referring to 0=not present; 1=mild degree; 2=moderate degree; 3=marked degree; 4=maximum degree, providing a total score range of 0-52, where higher score indicate greater symptom severity. A total score of 6-14 indicates mild anxiety; a total score of 15-28 indicates moderate anxiety; and a total score of 29-52 indicates severe anxiety.

### Sheehan Disability Scale (SDS) Score

The Sheehan Disability Scale (SDS) score is an unweighted composite of three self-reported items of family, work and social impairment in the previous week. Each item is preceded by a lead-in question: “The symptoms have disrupted your (work/studies; social life; family life/home responsibilities)”. Each domain is scored from 11 potential responses ranging from (0)=not at all; (1-3)=mildly; (4-6)=moderately; (7-9)=markedly; and (10)=extremely, i.e., higher scores correspond to greater disruption. See, e.g., Sheehan, et al., Int. Clin. Psychopharmacol. Suppl 3:89-95 (1996) and Sheehan and Sheehan, Int. Clin. Psychopharmacol. 23: 70-83 (2008).

Work is specified as paid work, unpaid volunteer work, or training and subjects have the option to skip this item if they have not worked/or studied at all in the last week for reasons unrelated to their disorder, for example, normal retirement. Subscale scores for work, social, and family disability are calculated separately. The three domains can be summarized to evaluate global functional impairment by adding the scores of each of the three domains, resulting in global SDS score ranges from (0)=unimpaired to (30)=highly impaired.

### Columbia Suicide Severity Rating Scale (C-SSRS)

The Columbia Suicide Severity Rating Scale (C-SSRS) is a measure used to identify and assess individuals at risk for suicide. Questions can be administered in an interview/clinical setting or answers can be self-reported. The C-SSRS measures four constructs: the severity of ideation, the intensity of ideation, behavior and lethality.

The CSSRS provides several questions directed to suicidal ideation that the subject answers with a “yes” or a “no.” Such questions are directed to a wish to be dead; non-specific active suicidal thoughts; active suicidal ideation with any methods (not a plan) without intent to act; active suicidal ideation with some intent to act; without a specific plan; and active suicidal ideation with specific plan and intent. Additionally, the CSSRS includes features that are rated by the subject to help assess the intensity of ideation. Such features include asking about the frequency (e.g., less than one a week, once a week, 2-5 times a week, daily or almost daily, and many times each day); duration (e.g., fleeting, less than an hour, 1-4 hours, 4-8 hours, more than 8 hours); controllability (e.g., easily able to control thoughts, can control thoughts with little difficulty, can control thoughts with some difficulty, can control thoughts with a lot of difficulty, unable to control thoughts, and does not attempt to control thoughts); deterrents (e.g., deterrents definitely stopped you from attempting suicide, deterrents probably stopped you, uncertain deterrent stopped you, deterrent most likely did not stop you, and deterrents definitely did not stop you); and reasons for ideation (e.g., completely to get attention, mostly to get attention, equally to get attention and to end/stop pain, mostly to end/stop pain, and completely to end/stop pain). The CSSRS can also include questions directed to suicidal behavior and an actual suicide attempt such as asking about if an attempt was made; asking if anything was done to cause harm to one's self, and asking if the subject has done anything dangerous where he or she could have died. Each “yes” answer is 1 and each “no” answer is 0 for a total score of 0-10, with higher scores indicating increased suicidal ideation.

### Clinical Opiate Withdrawal Scale (COWS)

The Clinical Opiate Withdrawal Scale (COWS) is a clinician-administered that rates eleven opiate withdrawal symptoms. The summed score for the complete scale can be used to help clinicians determine the stage or severity of opiate withdrawal and assess the level of physical dependence on opioids. See, e.g., Wesson and Ling, J. Psychoactive Drugs, 35(2):253-9 (2003).

The eleven items in COWS include: (a) resting pulse rate, (measured as 0 (≤80 BPM), 1, 2, 4); (b) sweating (measured as 0 (no report of chills or flushing), 1, 2, 3, 4); (c) restlessness (measured as 0 (able to sit still), 1, 3, 5); (d) pupil size (measured as 0 (pupils pinned or normal size for room light), 1, 2, 5); (e) bone or joint aches (measured as 0 (not present), 1, 2, 4); (f) runny nose or tearing (measured as 0 (not present), 1, 2, 4); (g) GI upset (measured as 0 (no GI symptoms), 1, 2, 3, 5); (h) tremor (measured as 0 (no tremor), 1, 2, 4); (i) yawning (measured as 0 (no yawning), 1, 2, 4); (j) anxiety or irritability (measured as 0 (none), 1, 2, 4); (k) gooseflesh skin (measured as 0 (skin is smooth), 3, 5).

The score for each item reflects the severity of the sign or symptom. Total scores of 5-12 indicate mild withdrawal; total scores of 13-24 indicate moderate withdrawal; total scores of 25-36 indicate moderately severe withdrawal; and total scores of more than 36 indicate severe withdrawal.

### Montgomery Asberg Depression Rating Scale (MADRS)

The Montgomery-Asberg Depression Rating Scale (MADRS) is a diagnostic questionnaire that can be used to measure the severity of a depressive episode in a subject. In some embodiments, the MADRS can be used to measure suicidal ideation. For example, the MADRS includes 10 items directed to the following: 1) apparent sadness (e.g., representing despondency, gloom and despair that is more than just ordinary transient low spirits that is reflected in speech, facial expression, and posture); 2) reported sadness (e.g., representing reports of depressed mood, regardless of whether it is reflected in appearance or not and can include low spirits, despondency or the feeling of being beyond help and without hope); 3) inner tension (e.g., representing feelings of ill-defined discomfort, edginess, inner turmoil, mental tension mounting to either panic, dread or anguish); 4) reduced sleep (e.g., representing the experience of reduced duration or depth of sleep compared to the subject's own normal pattern when well); 5) reduced appetite (e.g., representing the feeling of a loss of appetite compared with when-well); 6) concentration difficulties (e.g., representing difficulties in collecting one's thoughts mounting to an incapacitating lack of concentration); 7) lassitude (e.g., representing difficulty in getting started or slowness in initiating and performing everyday activities); 8) inability to feel (e.g., representing the subjective experience of reduced interest in the surroundings, or activities that normally give pleasure, and the ability to react with adequate emotion to circumstances or people is reduced); 9) pessimistic thoughts (e.g., representing thoughts of guilt, inferiority, self-reproach, sinfulness, remorse and ruin); and 10) suicidal thoughts (e.g., representing the feeling that life is not worth living, that a natural death would be welcome, suicidal thoughts, and preparations for suicide). Each item is rated from 0 to 6, with 0 reflecting that the subject is not at all as described by the item and 6 reflecting that the subject is extremely like what is described by the item. For example, for apparent sadness, a score of 0 can indicate that the subject does not display any sadness, whereas a score of 6 can indicate that the subject looks miserable all the time, e.g., the subject is extremely despondent. As another example, for suicidal thoughts, a score of 0 can indicate that the subject enjoys life or takes it as it comes; a score of 2 can indicate that the subject is weary of life and may have fleeting suicidal thoughts; a score of 4 can indicate that the subject feels he or she would probably be better off dead (e.g., suicidal thoughts are common, and suicide is considered as a possible solution, but without specific plans or intention); and a score of 6 can indicate that the subject has explicit plans for suicide when there is an opportunity (e.g., the subject has made active preparations for suicide).

Thus, the total score, after summation of each score for each item, is on a scale of 0 to 60. In some embodiments, a total score on the MADRS of about 0 to about 6 for the subject reflects the subject does not have symptoms related to depression; a score of about 7 to about 9 reflects that the subject has mild depression; a score of about 20 to about 34 reflects that the subject has moderate depression; and a score of about 34 to about 60 reflects that the subject has severe depression. MADRS is a clinician-rated scale.

### Clinical Global Impression-Severity (CGI-S)

The Clinical Global Impression-Severity (CGI-S) Scores is a subscale of Clinical Global Impression (CGI), a retrospective assessment that is a measure of symptom severity, treatment response and the efficacy of treatments in treatment studies, completed by the treating physician at baseline and at subsequent clinic visits to document any change in target symptoms documented at baseline. The CGI-S evaluates the presence of relevant symptoms, the frequency of their occurrence over the seven day rating timeframe, the intensity or severity of the symptoms, and the effect of the symptoms on functioning in major areas of the patient's life—work, home, school, and relationships. The typical time span rated for severity of illness is now or within the last week. See, e.g., Busner and Targum, Psychiatry, 4(7), 28-37 (2007).

The CGI-S asks the clinician one question: “Considering your total clinical experience with this particular population, how mentally ill is the patient at this time?” which is rated on the following seven-point scale: (1)=normal, not at all ill (symptoms of disorder not present past seven days); (2)=borderline mentally ill (subtle or suspected pathology); (3)=mildly ill (clearly established symptoms with minimal, if any, distress or difficulty in social and occupational function); (4)=moderately ill (overt symptoms causing noticeable, but modest, functional impairment or distress; symptom level may warrant medication); (5)=markedly ill (intrusive symptoms that distinctly impair social/occupational function or cause intrusive levels of distress); (6)=severely ill (disruptive pathology, behavior and function are frequently influenced by symptoms, may require assistance from others); (7)=among the most extremely ill patients (pathology drastically interferes in many life functions; may be hospitalized).

This rating is based upon observed and reported symptoms, behavior, and function in the past seven days. As symptoms and behavior can fluctuate over a week; the score should reflect the average severity level across the seven days.

### Symptoms of Major Depressive Disorder Scale (SMDDS)

The Symptoms of Major Depressive Disorder Scale (SMDDS) is a patient-reported outcome measure based on a 16-item scale addressing nine different domains of major depressive disorder (MDD) over the last seven days: (1) negative emotions/mood (4 items: sadness, hopeless/helpless, irritability, difficulty enjoying daily life (anhedonia); (2) anxiety (2 items: feeling overwhelmed, worry); (3) low energy (1 item: tiredness); (4) cognition (2 items: intrusive thoughts, poor concentration); (5) sleep disturbances (1 item: general sleep adequacy); (6) self-harm/suicide (1 item: life not worth living); (7) low motivation (2 items: lack of drive, no interest in activities); (8) sense-of-self (1 item: blame); and (9) eating behavior (2 items, scored as a single item: poor appetite, over eating). See, e.g., Bushnell, et al., Value Health, 22(8):906-915 (2019).

The SMDDS assesses changes in depressive symptom severity for adults (aged 18 years or older) who have been diagnosed and are being treated in an ambulatory setting for MDD. Because the symptom experience of MDD is chronic, the SMDDS measure asks respondents to report on the status of their MDD symptoms over the past seven days. Each item requires a response on a 5-point verbal rating scale using either (0)=not at all, (1)=a little bit, (2)=moderately, (3)=quite a bit, (4)=extremely (for intensity items); or (0)=never, (1)=rarely, (2)=sometimes, (3)=often, (4)=always (for frequency items). Higher scores indicate greater severity of MDD symptomology, with a total score range of 0-60.

### Self-Assessment of Treatment Experience (SATE) Score

The Self-Assessment of Treatment Experience (SATE) is a questionnaire with a one or two item self-report scale which provides additional information regarding an individual's subjective experience on depression, often since starting a new medication. This is a qualitative scale and the responses were recorded as ‘very much improved’, ‘much improved’, ‘improved’, ‘no change’, ‘worse’, ‘much worse’, and ‘very much worse’.

### Hamilton Anxiety Scale 6 (HAM-A6)

The Hamilton Anxiety Scale 6 (HAM-A6) is a clinician-rated scale including the 6-item Hamilton Anxiety Scale (HAM-A) subscale, which scale assesses the severity of different anxiety-related symptoms with a score range of 0 to 52. Each item is rated on a 5-point scale ranging from 0=not present; 1=mild degree; 2=moderate degree; 3=marked degree; 4=maximum degree. Each of the 14 items is rated by the clinician on a 5-point scale ranging from 0 (not present) to 4 (maximum degree). The 6 item subscale from HAM-A (HAM-A6) is a uni-dimensional, 6-item subscale derived from the original HAM-A. The HAM-A6 comprises of five psychic anxiety symptoms: anxious mood, psychic tension, fears, intellectual disturbances, and anxious behavior observed at the interview, as well as one somatic item, muscular tension, with a score range of 0 to 24. Higher scores represent more severe anxiety symptoms. See, e.g., Kent, et al., Prog. Neuropsychopharmacol. Biol. Psychiatry, 67:66-73 (2016) and Meoni, et al., J. Clin. Psychiatry, 62(11): 888-893 (2001).

### Structured Interview Guide for the Hamilton Anxiety Rating Scale (SIGH-A)

The Structured Interview Guide for the Hamilton Anxiety Rating Scale (SIGH-A) Score is a clinician-rated 14-item scale used to measure severity of different anxiety-related symptoms in subjects. Each of the 14 items is rated by the clinician on a 5-point scale ranging from 0 (not present) to 4 (maximum degree) with a total score range of 0 to 52 where higher score indicates worsening. Much like the HARS, the scale in SIGH-A relates to: (1) anxious mood; (2) tension; (3) fears; (4) insomnia; (5) ‘intellectual’; (6) depressed mood; (7) somatic symptoms; (8) sensory; (9) cardiovascular; (10) respiratory; (11) gastrointestinal; (12) genitourinary; (13) autonomic; and (14) observed behavior at interview. See, e.g., Shear, et al., Depress. Anxiety, 13(4): 166-78 (2001) and Rollman, et al., Arch. Gen. Psychiatry, 62(12): 1332-41 (2005).

### Prior Administration of Other Therapeutic Agents

In some embodiments, the subject was previously administered one or more therapeutic agents, i.e., antidepressant agents, prior to the administration of 1-(6-ethyl-8-fluoro-4-methyl-3-(3-methyl-1,2,4-oxadiazol-5-yl)quinolin-2-yl)-N-(tetrahydro-2H-pyran-4-yl)piperidin-4 amine, or a pharmaceutically acceptable salt thereof.

Some embodiments reference a time “prior to” administration of 1-(6-ethyl-8-fluoro-4-methyl-3-(3-methyl-1,2,4-oxadiazol-5-yl)quinolin-2-yl)-N-(tetrahydro-2H-pyran-4-yl)piperidin-4 amine, or a pharmaceutically acceptable salt thereof. The time prior to administration may be a particular time or range of time as indicated (e.g., about 30 minutes, about 1 hour, from about 1 day to about 1 week, 6 months, etc.), or it may be any time prior to administration if no particular time or range is specified.

In some embodiments, the subject has previously been administered a standard of care treatment for depression (including major depressive disorder) and the subject was not responsive to the previous therapy.

In some embodiments, the subject has previously been administered a standard of care treatment for anhedonia and the subject was not responsive to the previous therapy.

In some embodiments, the subject has previously been administered one or more antidepressants, and was not responsive to the previous therapy.

In some embodiments, the subject has previously been administered one or more antidepressants and was not responsive to the previous therapy. In some embodiments, the antidepressant is an atypical antidepressant, a selective serotonin reuptake inhibitor, a selective serotonin and norepinephrine reuptake inhibitor, a monoamine oxidase inhibitor, a kappa opioid receptor antagonist, or a selective norepinephrine reuptake inhibitor, and was not responsive to the previous therapy.

In some embodiments, the subject has previously been administered one or more selective serotonin reuptake inhibitors, such as citalopram, escitalopram, fluoxetine, fluvoxamine, paroxetine, and sertraline, and was not responsive to the previous therapy.

In some embodiments, the subject has previously been administered one or more selective serotonin and norepinephrine reuptake inhibitors, such as atomoxetine, desvenlafaxine, duloxetine, levomilnacipran, milnacipran, sibutramine, tramadol, and venlafaxine, and was not responsive to the previous therapy.

In some embodiments, the subject has previously been administered one or more monoamine oxidase inhibitors, such as moclobemide, rasagiline, selegiline, or safinamide, and was not responsive to the previous therapy.

In some embodiments, the subject has previously been administered one or more selective norepinephrine reuptake inhibitors, such as reboxetine, and was not responsive to the previous therapy.

In some embodiments, the subject has previously been administered one or more benzodiazepines, such as alprazolam, bromazepam, chlordiazepoxide, clonazepam, clorazepate, diazepam, flurazepam, lorazepam, oxazepam, temazepam, or triazolam, and was not responsive to the previous therapy.

In some embodiments, the subject has previously been administered one or more a kappa opioid receptor antagonists and was not responsive to the previous therapy. In some embodiments, the kappa opioid receptor antagonist is aticaprant.

In some embodiments, the subject exhibited no clinically meaningful response to a prior standard of care treatment for depression, as described herein.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. A Phase 2a, Randomized, Double-Blind, Placebo-Controlled Proof of Concept Study to Evaluate the Effects of Oral 1-[6-ethyl-8-fluoro-4-methyl-3-(3-methyl-1,2,4-oxadiazol-5-yl)quinolin-2-yl]-N-(oxan-4-yl)piperidin-4-amine Versus Placebo in Subjects with Major Depressive Disorder

This example describes a randomized Phase 2a, double blind placebo-controlled proof of concept study that evaluated the effects of orally administered 1-[6-ethyl-8-fluoro-4-methyl-3-(3-methyl-1,2,4-oxadiazol-5-yl)quinolin-2-yl]-N-(oxan-4-yl)piperidin-4-amine in comparison with placebo in subjects diagnosed with Major Depressive Disorder (MDD).

### Study Overview

### Objectives

The primary objective of this study was to establish Proof of Concept (POC) for 1-[6-ethyl-8-fluoro-4-methyl-3-(3-methyl-1,2,4-oxadiazol-5-yl)quinolin-2-yl]-N-(oxan-4-yl)piperidin-4-amine, a kappa opioid receptor antagonist, by evaluating the impact of 1-[6-ethyl-8-fluoro-4-methyl-3-(3-methyl-1,2,4-oxadiazol-5-yl)quinolin-2-yl]-N-(oxan-4-yl)piperidin-4-amine (hereinafter in Example 1 referred to as “the Study Compound”) relative to placebo on symptoms of MDD in adult subjects with MDD and symptoms of anhedonia and anxiety following 8 weeks of double-blind treatment as assessed by the Hamilton Depression Rating Scale (HAMD-17).

The secondary objectives were:
1. To evaluate the effects of the Study Compound on self-reported anhedonia in adult subjects with MDD.
2. To evaluate the effects of the Study Compound on anxiety-related symptoms in adult subjects with MDD.
3. To evaluate the effects of the Study Compound on functional impairment in adult subjects with MDD.
4. To evaluate the safety and tolerability of the Study Compound in adult subjects with MDD.

The exploratory objectives were:
1. To explore the effects of the Study Compound on an objective measure of hedonic tone in adult subjects with MDD.
2. To further explore the effects of the Study Compound on symptoms of depression and functional changes in adult subjects with MDD.
3. To explore if pre-existing genetic markers (e.g., markers for opioid receptor kappa 1 [OPRK1], such as those described herein), or biologic (i.e., acetyl-L-carnitine [LAC]) and digital (facial and speech recordings) biomarkers influence the effects of the Study Compound on symptoms of depression or anhedonia in adult subjects with MDD.
4. To explore the relationship between symptoms of depression and potential facial and speech biomarkers.
5. To characterize the pharmacokinetic (PK) profile of the Study Compound.

Number of Subjects: Approximately 204 subjects were randomized to the treatment phase.

Duration of Study: A subject's participation was estimated to be approximately 12 weeks.

Study Design: The Phase 2a study was an 8-week, randomized, double-blind, placebo-controlled, parallel-group, multicenter study to evaluate the effects of the Study Compound on symptoms of depression in adult subjects with MDD and symptoms of anhedonia and anxiety after 8 weeks of double-blind treatment. The study consisted of a 7- to 28-day screening period, an 8-week active treatment period (during which subjects received either the Study Compound or placebo), and a 4-week Safety Follow-up period.

Subjects signed an informed consent form (ICF) and then entered the screening period to ensure they met the required inclusion and exclusion criteria, including diagnosis and stability of depressive symptoms and absence of psychiatric and medical conditions that would preclude study participation. The 28-day screening period extended to 35 days (after consultation with and approval by the medical monitor), if necessary, for reasons including, but not limited to, discontinuation of a subject's current inefficacious medications or obtaining an ophthalmologic examination.

Re-screening a subject that previously screen failed the study was evaluated on a case-by-case basis.

After eligibility was confirmed by the medical monitor, and before randomization, baseline ophthalmologic examinations (standard and corneal specular microscopy) were performed. Subjects who met the Diagnostic and Statistical Manual of Mental Disorders Fifth Edition (DSM5) diagnostic criteria for MDD and all other eligibility criteria for the study were randomized in a 1:1 ratio to 1 of 2 treatment arms (placebo or the Study Compound once daily [QD]) until all subjects were randomized. Beginning at Visit 2 (Baseline), subjects received treatment with the Study Compound (80 mg) QD or placebo for 8 weeks. Scheduled visits took place every two weeks (Week 2, 4, 6, and 8).

During the Safety Follow-up, all subjects received a telephone call from study site personnel approximately 2 weeks after discontinuation of study drug for assessment of adverse effects (AEs), concomitant medication usage, and Columbia Suicide Severity Rating Scale (CSSRS); and at Week 12, subjects had corneal specular microscopy performed by a study site affiliated ophthalmologist. The overall duration in the study for each subject was approximately 14 to 16 weeks.

### Criteria for Inclusion

The following were the inclusion criteria. Subjects met each of the following inclusion criteria to be eligible:
1. Be an adult man or woman 18 to 65 years of age (inclusive) at informed consent.
2. Have a primary DSM-5 diagnosis of MDD, with prominent symptoms of anhedonia confirmed by Structured Clinical Interview for DSM-5 Disorders, Clinical Trials Version (SCID-5-CT).
   a. Have the current episode start at least 3 weeks prior to screening but no more than 12 months before the screening visit.
   b. Have not failed 2 or more courses of antidepressant treatment in the current episode.
   c. Have no more than a 3-point change in HAMD-17 between screening and baseline.
   d. Have sufficient history or an independent report to confirm that symptoms are causing functional impairment or clinically significant distress.
3. Meet the blinded-rule list based on clinical scale criteria.
4. Have a body mass index (BMI) of 18 to 40 kg/m² (inclusive).
5. Be medically stable based on medical history, vital signs, clinical laboratory tests, and 12-lead electrocardiogram (ECG) performed at screening and baseline.
6. Agree to the following birth control:
   a. Nonvasectomized men must agree to use a condom with spermicide, if sexually active during the study, until 90 days after the last dose of study drug administration. No restrictions are required for a vasectomized man, provided his vasectomy was performed 4 months or more prior to the first dose of study drug. A man who has been vasectomized less than 4 months prior to the first dose of study drug must follow the same restrictions as a nonvasectomized man. Additionally, men must refrain from sperm donation during study treatment and for at least 90 days following the last dose of study drug.
   b. Women of child-bearing potential (women not surgically sterilized and between menarche and 2 years postmenopausal) must have a negative serum pregnancy test at screening and a negativeurine pregnancy test at enrollment and agree to use reliable birth control. Women were considered surgically sterile, if they had tubal ligation, bilateral salpingo oophorectomy, or a hysterectomy.
   c. Or engaged exclusively in a non-heterosexual relationship.
7. Willing and able to give written informed consent to participate.
8. Able to understand and comply with instructions in English.

### Exclusion Criteria

Subjects were excluded from the study if they met any of the following criteria:
1. Have a history of any of the following DSM-5 disorders within the specified timeframe:
   a. Currently or in the past year: diagnosis of personality disorder, attention deficit disorder/attention deficit hyperactivity disorder, anorexia nervosa, or bulimia nervosa. Subjects with comorbid generalized anxiety disorder, social anxiety disorder, or panic disorder for whom MDD is considered the primary diagnosis were not excluded.
   b. Lifetime: diagnosis of bipolar 1 or 2, schizophrenia, obsessive compulsive disorder, or post-traumatic stress disorder.
2. Have a history of substance or alcohol use disorder (AUD), per DSM-5 criteria, within the past year.
3. Are actively suicidal (e.g., any suicide attempts within the past 12 months) or are at serious suicidal risk as indicated by any current suicidal intent, including a plan, as assessed by the Columbia Suicide Severity Rating Scale (C-SSRS) (score of “YES” on suicidal ideations item 4 or 5 within 3 months prior to Visit 1 [Screening]) and/or based on clinical evaluation; or are homicidal.
4. Have a history or signs of Cushing's disease, Addison's Disease, primary amenorrhea or other evidence of significant disorders of the hypothalamus-pituitary-adrenal axis.
5. Have any other clinically significant medical or psychiatric condition or circumstance prior to randomization that could affect subject safety, preclude evaluation of response, interfere with the ability to comply with study procedures, or prohibit completion of the study, such as acute stress disorder, adjustment disorder, impulse control disorder, uncontrolled diabetes mellitus, renal or hepatic impairment, coronary artery disease, evidence of significant active cardiac, respiratory, or hematologic disease, cancer with <5-year remission (basal cell carcinoma is not excluded), chronic pain, fibromyalgia, gastric bypass, lap band placement, or any other significant gastrointestinal condition.
6. Have had prior seizures (other than remote history of childhood febrile seizure) or other condition that would place the subject at increased risk of seizures or is taking anticonvulsants for seizure control.
7. Have a history of serious head injury (e.g., skull fracture, cerebral contusion, or trauma resulting in prolonged unconsciousness), intracranial neoplasm, or hemorrhage.
8. Have ever had electroconvulsive treatment, vagal nerve stimulation, or treatment with ketamine or esketamine for MDD.
9. Have initiated transcranial magnetic stimulation, psychotherapy (such as Cognitive Behavioral Therapy) or have had a change in psychotherapy, or other non-drug therapies (such as acupuncture or hypnosis) within 4 weeks prior to Visit 1 (Screening) or at any time during the acute phase of the study.
10. Have a visual or physical motor impairment that could interfere with subject's ability to perform study assessments.
11. Have alanine aminotransferase (ALT) or aspartate aminotransferase (AST) levels ≥2×upper limit of normal (ULN) or a bilirubin level 1.5×ULN unless due to a documented history of Gilbert's syndrome.
12. Have an estimated glomerular filtration rate (eGFR)≤60 mL/min/1.73 m² as calculated by the Chronic Kidney Disease Epidemiology Collaboration [CKD-EPI]2009 creatinine equation at Visit 1 (Screening).
13. Have a positive hepatitis C virus (HCV) antibody (Ab), hepatitis B surface antigen (HBs Ag), hepatitis A virus (HAV) IgM antibody (HAV-Ab [IgM]) or human immunodeficiency virus (HIV) test at Visit 1 (Screening).
14. Have a thyroid-stimulating hormone (TSH) level of <0.9×lower limit of normal (LLN) or >1.2×ULN on or off stable treatment for hyperthyroidism or hypothyroidism; if TSH is abnormal, evaluate reflex Free T3 and Free T4.
15. Have any other clinically significant abnormalities (significant would include laboratory deviations requiring acute medical intervention or further medical evaluation) in laboratory results at screening, including clinical chemistries, hematology, and urinalysis, and any clinical information that should preclude a subject's participation at study entry.
16. Exclusionary ECG abnormalities obtained at Visit 1 (Screening) or Visit 2 (Baseline) are QT interval corrected using Fridericia's formula (QTcF) >450 msec in males or >470 msec in females, complete bundle branch block, evidence of myocardial infarction or ischemia, and predominantly nonsinus conducted rhythms.
17. Have a positive urine drug screen for amphetamines, barbiturates, cocaine, methadone, opioids, propoxyphene, tetrahydrocannabinol (THC), phencyclidine, or a positive blood alcohol level assessed by breathalyzer at Visit 1 (Screening) and Visit 2 (Baseline). For occasional (1 to 2 times per month maximum) cannabis users only, 1 retest is allowed and subject must agree to abstain from use for the duration of the study; a positive second test is exclusionary.
18. Have any use, by history, of Salvinorin A.
19. Use of the following concomitant medications:
   a. Psychoactive medication including stimulants, benzodiazepines and anxiolytics, oral antipsychotics, mood stabilizers/anticonvulsants (carbamazepine, lamotrigine, etc.), lithium, antidepressants, S-adenosylmethionine, melatonin, agomelatine, and hypnotics/sedatives within 5 half-lives or 14 days (whichever is longer) of Visit 2 (Baseline).
   b. Fluoxetine and irreversible monoamine oxidase inhibitors within 4 weeks of Visit 2 (Baseline) depot antipsychotics within 2 months of Visit 2 (Baseline).
   c. Opioid agonists and antagonists.
20. Are currently taking or have taken within 5 half-lives of Visit 2 (Baseline) any medications or supplements that are moderate or strong inhibitors or inducers of cytochrome P450 (CYP) 3A4, on a diet likely to modulate CYP3A4 activity, or are taking substrates of P-glycoprotein (P-gp) with narrow therapeutic windows (e.g., digoxin).
21. Are women who are either pregnant or breastfeeding.
22. Have participated (received study treatment) in a clinical study or any other type of medical research judged to be scientifically or medically incompatible with this study within 30 days prior to Visit 1 (Screening).
23. Have participated in multiple interventional clinical studies such that the subject is not a suitable candidate for participation.
24. Have previously completed or withdrawn from this study or any other study investigating the Study Compound.
25. Has any of the following:
   a. Useful vision in only 1 eye from a pre-existing ophthalmic disease or amblyopia;
   b. A corneal transplant in either eye;
   c. Corneal dystrophy or family history of corneal dystrophy;
   d. Severe dry eye syndrome [keratitis sicca];
   e. Will not or cannot cooperate with ophthalmic examination requiring pupillary dilation (includes history of severe adverse reaction to mydriatic agents or untreated narrow angle glaucoma).
Note: The following ocular disorders were allowed: cataracts, prior cataract surgery, glaucoma (narrow angle glaucoma was allowed if definitively treated with laser peripheral iridectomy), macular degeneration, or ocular changes associated with diabetes mellitus or multiple sclerosis.

### Investigational Product, Dose, and Mode of Administration

A starting dose of the Study Compound (80 mg (1 capsule)) QD at Visit 2 (Baseline), orally and this dose was maintained for the subsequent 8 weeks.

### Reference Therapy, Dose, and Mode of Administration

Placebo was assigned as 1 capsule QD at Visit 2 (Baseline), orally, and was maintained for the subsequent 8 weeks. Placebo capsules consisted of inactive ingredients and looked identical to the Study Compound.

### Study Assessments

### Efficacy:

HAMD-17
Clinical Global Impression of Severity (CGI-S)
Clinical Global Impression of Improvement (CGI-I)
Snaith-Hamilton Pleasure Scale (SHAPS)
Hamilton Anxiety Rating Scale (HAM-A)
Hospital Anxiety and Depression Scale (HADS) subscales (i.e., anxiety subscale [HADS-A] and depression subscale [HADS-D])
Sheehan Disability Scale (SDS)
State-Trait Anxiety Inventory (STAI) subscales (i.e., state anxiety subscale score [S-anxiety] and trait anxiety subscale score [T-anxiety])
Probabilistic Reward Task (PRT) (optional)
Patient Health Questionnaire-9 (PHQ-9)

### Safety:

Safety assessments included adverse events (AEs), serious adverse events (SAEs), adverse events of special interest (AESIs), vital signs, weight and BMI, clinical laboratory tests, physical, ECG, and ophthalmologic examinations (standard and corneal specular microscopy), C-SSRS, and Clinical Opiate Withdrawal Scale (COWS).

### Pharmacogenetics and Biomarkers

Blood samples for pharmacogenetics (e.g., genetic markers for OPRK1 such as those described herein), and blood-based biomarker (i.e., LAC); digital biomarkers (facial and speech recordings) were also obtained; all assessments were optional.

Pharmacokinetics: Plasma concentrations

### Study Endpoints

### Primary Efficacy Variable

The primary efficacy endpoint was the change from baseline to Week 8 on the HAMD-17 score in the Efficacy population.

This instrument was completed based on a semi-structured interview (Structured Interview Guide for the Hamilton Depression Rating Scale [SIGH-D], Williams J B W. A Structured Interview Guide for the Hamilton Depression Rating Scale. Arch Gen Psychiatry. 1988; 45(8):742-747. doi:10.1001/archpsyc.1988.01800320058007). The 17-item HAM-D comprised individual ratings related to the following symptoms: depressed mood (sadness, hopeless, helpless, worthless), feelings of guilt, suicide, insomnia (early, middle, late), work and activities, retardation (slowness of thought and speech; impaired ability to concentrate; decreased motor activity), agitation, anxiety (psychic and somatic), somatic symptoms (gastrointestinal and general), genital symptoms, hypochondriasis, loss of weight, and insight. The HAMD-17 scoring ranged from 0 to 52 with individual items ranging from 0-4 points and 0-2 points where higher scores corresponded to higher levels of symptom severity.

### Secondary Efficacy Variables

Secondary endpoints included the following:
HAMD-17 response rate at Week 4 and Week 8. The response rate was the percentage of subjects with ≥50% decrease from baseline in HAMD-17 score.
CGI-I. CGI-I score assessed at each post baseline timepoint compared to baseline. The CGI-I scale (Guy, 1976) is a clinician-rated instrument that measures the improvement of the subject's symptoms. It is a 7-point scale, where a score of 1 indicated that the subject was “very much improved,” a score of 4 indicated that the subject had experienced “no change,” and a score of 7 indicated that the subject was “very much worse.” For analysis purposes, a CGI-I response indicating symptom improvement was defined as “much improved” or “very much improved”.
SHAPS. Change from baseline to each timepoint assessed in SHAPS score. The SHAPS is a 14-item self-report instrument, which measures anhedonia (Snaith R P, Hamilton M, Morley S, et al. A scale for the assessment of hedonic tone. The Snaith-Hamilton Pleasure Scale. Br J Psychiatry. 1995; 167(1):99-103.). Each of the 14 items had a set of 4 responses, 2 of which endorsed agreement (Definitely/Strongly Agree, Agree) and 2 of which endorsed disagreement (Disagree, Strongly Disagree). There are two scoring methods. Both methods were used for analyses.
SHAPS Original Total Score (0 to 14): The original method assigned a score of 1 to either of the disagreement responses (Disagree, Strongly Disagree), and a score of 0 to either of the agreement responses (Definitely/Strongly Agree, Agree). Therefore, original scores on the SHAPS ranged from 0-14 with higher scores corresponding to higher levels of anhedonia.
SHAPS Total Score (14 to 56): The second method derived a total score by summing the item responses, where Definitely/Strongly Agree responses scored 1 point, Agree responses scored 2 points, Disagree responses scored 3 points, and Strongly Disagree responses scored 4 points. Therefore, scores on the SHAPS ranged from 14 to 56 by the second method, with higher scores corresponding to higher levels of anhedonia.
HADS. Change from baseline to each timepoint assessed in HADS total score and HADS subscales (i.e., anxiety subscale [HADS-A] and depression subscale [HADS-D] scores). The HADS measures levels of anxiety and depression without regards to somatic symptoms (Zigmond A S, Snaith R P. The hospital anxiety and depression scale. Acta Psychiatr Scand. 1983; 67(6):361-70). This self-report scale consists of 14 items. Seven of the items were used to evaluate anxiety and 7 to evaluate depression. Each item on the questionnaire was scored from 0 to 3. Therefore, the anxiety subscale (HADS-A) and the depression subscale (HADSD) each ranged from 0 to 21.
HAM-A. Change from baseline to each timepoint assessed in HAM-A total score. This instrument was completed based on a semi-structured interview (Structured Interview Guide for the Hamilton Anxiety Rating Scale [SIGH-A], Williams J B W. A Structured Interview Guide for the Hamilton Depression Rating Scale. Arch Gen Psychiatry. 1988; 45(8):742-747. doi:10.1001/archpsyc.1988.01800320058007). The scale consists of 14 items. Each item was rated on a scale of 0 (feeling not present) to 4 (very severe prevalence of the feeling). The HAM-A total score was the sum of the 14 items and the score ranged from 0 to 56, where higher scores indicated more severe anxiety.
SDS. Change from baseline to each timepoint assessed in SDS scores. SDS is a brief self-rated tool where the subject rated how symptoms of depression had disrupted the 3 domains (related to work/school, social, and family life) on an anchored 10-point visual analogue scale (Sheehan D V. The Sheehan Disability Scales. In: The Anxiety Disease and How to Overcome It. Sheehan D V, ed. New York, NY: Charles Scribner and Sons; 1983:151). The SDS total score was the sum of the 3 individual item scores where higher scores indicated more severe disability related to MDD symptoms.
CGI-S. Change in CGI-S from baseline to Week 8. The CGI-S is a clinician-rated instrument that measures the severity of depression at the time of assessment. This rating was based upon observed and reported symptoms, behavior, and function in the past 7 days. The score reflected the average severity level across the 7 days. The CGI-S was scored on a 7-point scale, where a score of 1 indicated that the subject was “normal, not at all ill”, a score of 4 indicated that the subject was “moderately ill,” and a score of 7 indicated that the subject was “among the most extremely ill subjects” (Guy W. ECDEU assessment manual for psychopharmacology-revised. Rockville, MD: National Institute of Mental Health, Psychopharmacology Research Branch, Division of Extramural Research Programs. 1976. 217-22).
Assessment of safety through the occurrence of treatment-emergent AEs (TEAEs), SAEs, and AESIs; change from baseline in vital signs measurements, body weights, BMI, clinical laboratory test results, C-SSRS scores, COWS scores; physical examination findings, ECG results, and ophthalmologic examination findings.

### Exploratory Efficacy Variables

Exploratory endpoints included the following:
PRT. Change from baseline to each timepoint assessed in performance on the PRT. The PRT is a measure of reward learning consisting of 300 trials in 3 blocks of 100 in which subjects were asked to respond to two hardly distinguishable cues, of which one was more frequently rewarded. All performance measures, including response bias for the rewarded category (Pizzagalli D A, Iosifescu D, Hallettb L A, et al. Reduced Hedonic Capacity in Major Depressive Disorder: Evidence from a Probabilistic Reward Task. J Psychiatr Res. 2008; 43(1):76-87), discriminability (subject's ability to differentiate between the two cues), reaction time, and accuracy (hit rates) were computed and provided by the vendor in the source data for each block and as an overall total. Response bias reflected the subject's propensity to select the rich option over the lean option irrespective of trial type, with higher scores indicating greater reward responsiveness. Discriminability reflected the subject's ability to perceptually distinguish between the two stimuli, with lower scores indicating greater task difficulty.
STAI. Change from baseline in STAI subscales (i.e., state anxiety subscale score [S-anxiety] and trait anxiety subscale score [T-anxiety]). Each subscale contained 20 items addressing somatic, affective, and cognitive aspects. State anxiety items included: “I am tense; I am worried” and “I feel calm; I feel secure.” Trait anxiety items include: “I worry too much over something that really doesn't matter” and “I am content; I am a steady person.” All items were rated on a 4-point scale (e.g., from “Almost Never” to “Almost Always”) and each subscale ranged from 0 to 80. Higher scores indicated greater anxiety.
PHQ-9. Change from baseline to each timepoint assessed in the PHQ-9 total score and key individual scores. scores. The PHQ-9 is a self-administered assessment scale for the evaluation of depression symptoms over the past 2 weeks. The PHQ-9 is a reliable and valid measure of depression and depression severity (Beard 2016). The questionnaire consisted of 9 items and each item was scored from 0 to 3. Subjects were asked how often they had been bothered by symptoms over the past 2 weeks (Not at all [0], Several days [1], More than half the time [2], or Nearly every day [3]). Higher scores indicated higher levels of depression.
HAMD-6. The HAMD-6 score was derived to be equal to the sum of the following 6 items in the HAMD-17: item 1-depressed mood; item 2-work and activities; item 9-somatic symptoms, general; item 10-feelings of guilt; item 12-anxiety psychic; and item 17-retardation. Higher scores indicating greater severity of depression symptoms.
The following exploratory endpoints were not included in this statistical analysis plan (SAP): pharmacogenetics (e.g., genetic markers for OPRK1) and biologic and digital biomarkers. Digital markers include facial and speech recording which were self-administered via an iPhone®-based application. Subjects completed a short facial and speech recording (approximately 3-5 minutes in duration), which included reading prompts and questions to be answered aloud. Subjects video-recorded their face and speech as they followed the prompts. Facial, speech, and linguistic features were extracted from files for analysis.
Individual plasma concentrations of the Study Compound

All efficacy endpoints were also summarized by visit, including change from baseline information.

### Statistical Methods

SAS® software, Version 9.4 or higher, was used to analyze data. Subjects were analyzed according to randomized treatment assignment for efficacy assessments and according to treatment received for safety assessments.

### Baseline Definition

For efficacy and safety analyses presented by study visit, the baseline value was defined as the last measurement prior to the start of study drug administration.

### Multiple Comparisons/Multiplicity

The prespecified primary efficacy analysis was conducted using a two-sided test at the α=0.05 level of significance. All other analyses were also conducted with no adjustments for multiple comparisons.

### Analysis Populations

The analysis populations are defined as follows:
The Safety population: The Safety population included all subjects who received study drug and were used for summaries of safety parameters. Subjects were analyzed according to treatment received.
The Efficacy population: The Efficacy population included all randomized subjects who received at least 1 dose of study drug, had a baseline HAMD-17 assessment total score, and had at least 1 post-baseline HAMD-17 assessment total score. Subjects were analyzed according to randomized treatment assignment. This population was used for the primary analysis and for all other efficacy analyses.
The Per-Protocol (PP) population: The PP population included subjects in the efficacy population except those who experienced an important protocol deviation that was determined to substantially impact efficacy analysis, or did not have a HAMD-17 score at Week 8 analysis visit, or whose compliance at the end of the study was less than 70%. Subjects were analyzed according to randomized treatment assignment. This population was used for supportive efficacy analyses. The list of important protocol deviations for PP population was classified by the sponsor before the final database lock and the unblinding of the study.

### Efficacy Evaluation

All efficacy summaries were produced on the Efficacy Population; select efficacy summaries (including HAMD-17, response rate of HAMD-17, subgroup analysis of HAMD-17 (<22 vs ≥22), SHAPS, CGI-I) were also produced on the PP Population. A data listing of subjects excluded from the Efficacy Population or PP Population, to include the reason for exclusion, was presented. All data listings were listed for all randomized or treated subjects.

Demographic variables including age, sex, ethnicity and race were summarized by treatment group and over all subjects combined for the Safety, Efficacy, and PP Populations. Age at the time of informed consent, as collected on the eCRF, was summarized using descriptive statistics. Sex, ethnicity, and race were summarized with the number and percentage of subjects in each parameter category (including the number missing).

Baseline characteristics included height, weight, BMI, baseline CGI-S score, HAMD-17 score, SHAPS scores by both scoring methods, HAM-A score, STAI subscale scores, HADS subscale and total scores, PHQ-9 total score, SDS total score, duration of current depressive episode, time since first diagnosis of depression, MDD medication use for the current episode of MDD prior to the start of study drug administration, nonpharmacological procedures for the current episode of MDD, and medical and psychiatric history other than MDD. Height, weight, BMI, baseline CGI-S score, HAMD-17, SHAPS scores by both scoring methods, HAM-A score, STAI subscale scores, HADS total and subscale scores, PHQ-9 total score, SDS total score, duration of current depressive episode, and time since first diagnosis of depression were summarized using descriptive statistics by treatment group and over all subjects combined for the Safety, Efficacy, and PP Populations.

Medications for the current episode of MDD prior to the start of study drug administration were coded using World Health Organization Drug Classification (WHODrug) for Drug Statistics Methodology (B3 Global) September 2019 version. For the MDD medication summary, the number and percentage of subjects receiving any medication were summarized by treatment group, as were the number and percentage receiving any medication by Anatomical Therapeutic Chemical (ATC) drug class and generic drug name. Subjects reporting use of more than one medication at each level of summarization (any medication received, ATC class, and generic drug name) were counted only once. ATC class terms were displayed by descending order of incidence, as were generic drug names within each ATC class.

Medical and psychiatric history other than MDD were presented only for the Safety Population. Medical and psychiatric history conditions were coded using the Medical Dictionary for Regulatory Activities (MedDRA, version 22.1). Medical and psychiatric history were summarized using frequency counts and percentages by treatment group, system organ class and preferred term.

All other baseline characteristics except MDD non-pharmacologic procedures were summarized by treatment group and over all subjects combined for the Safety, Efficacy, and PP Populations. Demographic and baseline characteristic information was listed for all randomized or treated subjects.

### Primary Efficacy Endpoint Analysis Methods

The primary efficacy endpoint was the change from baseline to Week 8 on the HAMD-17 score. The estimand for the primary efficacy endpoint was the difference in means between treatment groups in the change of HAMD-17 scores from baseline to Week 8 for all subjects in the Efficacy population. For efficacy analyses, baseline was defined as the latest measurement prior to the first administration of study drug. The null hypothesis tested was that there is no difference between the Study Compound and placebo:
H₀: μ_{A}=μ_{B};
Where μ_{A} and μ_{B} represent the mean values for the Study Compound and placebo, respectively. The alternate hypothesis tested was that the treatment group means differ:
H1: μ_{A}≠μ_{B};

The analysis was conducted using a mixed-models repeated measures (MMRM) with change from baseline in HAMD-17 score as the dependent variable and included treatment group, week, and week by treatment group interaction as factors, and baseline HAMD-17 score as covariate. Variance estimation was based on an unstructured covariance matrix. If the unstructured covariance matrix resulted in a lack of convergence, the heterogeneous autoregressive variance-covariance structure was used.

The primary analysis compared the groups at Week 8. The primary results obtained from the model was the estimated treatment difference at Week 8. Significance was assessed based on a two-sided test at the α=0.05 level of significance. In addition, the estimated treatment difference at Week 4 was provided. The model-based least square (LS) means, standard errors, 95% CIs, and 2-sided p-values were reported.

In addition, descriptive summary of HAMD-17 and each individual item was generated by treatment group and by visit.

### Sensitivity Analyses of the Primary Efficacy Endpoint

Sensitivity analyses were used to investigate the impact of important protocol deviations or missing data for the primary efficacy endpoint assessment (i.e., HAMD-17 total score). The primary efficacy analysis was repeated using:
The per-protocol population.
When ≥10% of subjects had missing data for the primary efficacy endpoint assessment (i.e., HAMD-17 total score), the following was conducted:
Rubin's rules for multiple imputation (MI) with SAS Proc MI and MIANALYSIS for the efficacy population.
Last Observation Carried Forward (LOCF) Imputation technique, i.e., the last observed non-missing value was used to fill in missing values at a later point in the study, regardless of when the missing value occurred. The imputed dataset generated with the LOCF technique was used as input data in the ANCOVA. The efficacy population was used.

### Subgroup Analyses of the Primary Efficacy Endpoint

The primary efficacy endpoint (change from baseline to Week 8 in HAMD-17 total score) was summarized using the following subgroups:
Gender (male and female)
Baseline HAMD-17 scores: <22 vs ≥22
by baseline SHAPS Total Score (14 to 56) (<median and ≥median) and SHAPS Original Total Score (0 to 14) (<median and ≥median).
by baseline STAI S-anxiety score (<median and ≥median) and baseline STAI T-anxiety score (<median and ≥median).

Summaries by subgroup were only produced when there were at least 20 subjects in the category of interest. Additional subgroup analyses were performed post-hoc, as appropriate.

The primary endpoint (HAMD-17 total score) was also summarized in subgroups of the Efficacy Population that included all subjects screened on or before Mar. 16, 2020 (labeled as PreCOVID) versus the subjects screened after Mar. 16, 2020 (labeled as PostCOVID)

### Secondary Efficacy Endpoint Analysis Method

Secondary endpoints including the change from baseline to each timepoint assessed for comparisons between the Study Compound and placebo for the measures listed below, were analyzed using an MMRM model for treatment group, week, and week by treatment group interaction as fixed factors and a covariate adjustment for baseline score. For CGI-I, the baseline CGI-I score was not included as a covariate, as the baseline assessment of CGI-I referred to the comparison with screening visit. For all other measures, the baseline score included as a covariate was the baseline score for that measure. Variance estimation was based on an unstructured covariance matrix unless there was a lack of convergence in which case a heterogeneous autoregressive variance-covariance structure was used.
CGI-I at each post-baseline visit
Change in SHAPS from baseline at each post-baseline visit
Change in HADS total score and HADS subscales: HADS-A and HADS-D from baseline at each post-baseline visit
Change in HAM-A total score from baseline at each post-baseline visit
Change in SDS from baseline at each post-baseline visit

The response rates (the percentage of subjects with ≥50% decrease in HAMD-17 from baseline to Week 4 and Week 8) were compared between the Study Compound and placebo-treated groups. The response rates, response rate difference and corresponding 95% Wald CI, and Cochran-Mantel-Haenszel p-value were reported.

The change at Week 8 from baseline in CGI-S was estimated using an analysis of the covariance (ANCOVA) model with main effects for treatment group and a covariate adjustment for baseline CGI-S score. The model-based LS means, standard errors, 95% CIs, and 2-sided p-values were reported.

For SDS, analysis was performed for the three SDS items separately and the SDS total score as the sum of the individual domains. Note that the work/school domain might be not applicable for all subjects and therefore the analysis of SDS total score was performed for the subjects with all 3 domain scores and the checkbox of “No work or school” was NOT marked. In addition, a sensitivity analysis of SDS total score was conducted for all subjects, where the SDS total score was calculated as (sum of social and family life)*30/20 for the subjects for whom the checkbox of “No work or school” was marked.

For CGI-I, an additional analysis was performed to compare the symptom improvement rate, i.e., the percentage of subjects with responses at each post-baseline visit of ‘much improved’ or ‘very much improved’ (i.e., CGI-I score ≤2) between the Study Compound and placebo. The symptom improvement rate, improvement rate difference and corresponding 95% Wald CI, and Cochran-Mantel-Haenszel p-value was reported.

For SHAPS, the analyses was performed for the SHAPS total scores based on both methods of scoring (SHAPS Total Score [14 to 56] and SHAPS Original Total Score [0 to 14]).

In addition, descriptive summaries of each secondary efficacy variable were generated by treatment group and by visit.

### Subgroup Analyses of the Secondary Efficacy Endpoints

The secondary efficacy endpoints HAM-A and SHAPS were summarized using the following subgroups:
HAM-A total score was presented by baseline SHAPS score (<median and ≥median).
SHAPS score (both SHAPS Total Score [14 to 56] and SHAPS Original Total Score [0 to 14]) was presented by baseline STAI S-anxiety score (<median and ≥median) and baseline STAI T-anxiety score (<median and ≥median).

Summaries by subgroup were only produced if there were at least 20 subjects in the category of interest. Additional subgroup analyses were performed post-hoc, as appropriate.

### Exploratory Efficacy Endpoint Analysis

### PRT Measures

The PRT is a measure of reward learning consisting of 300 trials in 3 blocks of 100. All PRT outcome measures including response bias for the rewarded category, discriminability, reaction time, and accuracy were computed and provided by the vendor in the source data for each block and as an overall total. The response bias and discriminability measures also had log-linear adjusted values provided. The adjusted values for response bias and discriminability and original values for reaction time and accuracy at each block were utilized in the analyses. PRT data was processed through a QC check to determine if they were usable for analysis per Pizzagalli (2008).

For response bias and discriminability, a block (1,2,3) by treatment (the Study Compound, placebo) ANCOVA model was performed to compare the change from baseline values between two treatment arms for each block and other metrics of interest (i.e., average of all blocks, average of Block 2 and Block 3, and Block 3-Block 1). The model included treatment group, block, and block by treatment interaction as fixed factors and a covariate for baseline value of corresponding outcome. LS means with corresponding SEs, 95% CIs for differences, and p-values were also presented.

For reaction time and accuracy, a block (1,2,3) by treatment (the Study Compound, placebo) ANCOVA model was performed to compare the change from baseline values between two treatment arms for each block and other metrics of interest (i.e., average of all blocks, average of Block 2 and Block 3, and Block 3-Block 1). The model included treatment group, block, block by treatment interaction, and stimulus as fixed factors and a covariate for baseline value of corresponding outcome. LS means with corresponding SEs, 95% CIs for differences, and p-values were also presented.

In addition, descriptive summary statistics was generated by treatment group, stimulus (if applicable), block, and visit.

### STAI Subscales

The change at Week 8 from baseline in STAI subscales (i.e., S-anxiety and T-anxiety) was estimated using an ANCOVA model with main effects for treatment group and a covariate adjustment for baseline score. The model-based LS means, standard errors, 95% CIs, and p-values were reported.

In addition, descriptive summary was generated by treatment group and by visit.

### PHO-9

The PHQ-9 total score and PHQ-9 item #1a (little interest or pleasure in doing things), including the change from baseline to Week 4 and Week 8 for comparisons between the Study Compound and placebo, were analyzed using the same statistical methodologies as applied to the primary efficacy endpoint, described above.

Correlations between PHQ-9 scores and other endpoints were explored by calculating Spearman's correlation coefficient and the corresponding confidence interval between the following scores:
Correlation between baseline score on PHQ-9 item #1a (Little interest or pleasure in doing things) and baseline SHAPS Total Score (14 to 56)
Correlation between baseline score on PHQ-9 item #1a (Little interest or pleasure in doing things) and baseline SHAPS Original Total Score (0 to 14)
Correlation between baseline PHQ-9 total score and baseline HAMD-17 total score
Correlation between baseline PHQ-9 total score and baseline SHAPS Total Score (14 to 56)
Correlation between baseline PHQ-9 total score and baseline SHAPS Original Total Score (0 to 14)

Other correlations between PHQ-9 and other endpoints were explored post-hoc. In addition, descriptive summary was generated by treatment group and by visit.

### Other Exploratory Analyses

For HAMD, an additional analysis was performed to compare the HAMD-6 score between the Study Compound and placebo by the same type of MMRM model as the primary endpoint. The estimated treatment difference at Week 4 and Week 8 was provided. The model-based least square (LS) means, standard errors, 95% CIs, and 2-sided p-values were reported.

### Plasma Concentrations

A descriptive summary by visit and time point was generated of plasma concentrations of the Study Compound by treatment group based for the Safety Population. A listing of plasma concentrations of the Study Compound was also generated.

### Safety Analyses

Safety data, including AEs, safety laboratory results, physical examination results, vital signs, suicidality, COWS scores, ECGs, and ophthalmologic examination findings was summarized by treatment group and/or listed. Safety analysis was carried out for the Safety Population, to include all subjects who received at least one dose of study drug. Safety analyses included data for each subject during the treatment-emergent period (from first dose of study drug to 30 days after last dose) except for the summary of AESIs. Subjects who did not complete the study, for whatever reason, had all available data up until the time of termination included in the analysis.

### Determination of Sample Size

The study sample size was determined based on a 2-sided t-test for the primary endpoint of change from baseline to Week 8 in the HAMD-17 score. The study was powered at 84% to detect a difference in means of 3 points between the Study Compound and Placebo subjects, at a 1:1 randomization ratio, using an alpha level of 0.05, and a standard deviation (SD) of 5.65. A 30% dropout is assumed. Using these assumptions, the approximate number of subjects in each arm was 90 for a total of 180 subjects.

Previously, an additional 24 subjects were added to the original planned study sample size of 120 (for a total of 144 subjects) to account for the 24 subjects who were enrolled at the time enrollment was temporarily suspended due to the spread of COVID-19. The data for these 24 subjects might have been impacted by safety procedures instituted at study sites during the suspension, and increasing the sample size allowed for a more homogeneous evaluation of the originally planned 120 subjects enrolled once sites reopened after standardized procedures were instituted.

SCHEDULE OF PROCEDURES AND ASSESSMENTS

| | Period | | | | | | | |
|---|---|---|---|---|---|---|---|---|
| | Screening | Baseline | Study Drug Treatment | | | | | |
| | Visit | | | | | | | |
| | 1^{b} | | 3 | 4 | 5 | 6 | Safety Follow-up | |
| | Days −7 | 2 | Day 15 | Day 29 | Day 43 | Day 57/ EOT^{c} | | Ocular |
| | to −28 | Day 1 | (±2 d) | (±2 d) | (±2 d) | (±2 d) | TC^{a} | Exam |
| Procedure and Assessments | NA | Week 0 | Week 2 | Week 4 | Week 6 | Week 8 | Week 10 | Week 12 |
| Informed consent | X | | | | | | | |
| Inclusion/exclusion criteria | X | X^{d} | | | | | | |
| Medical/psychiatric history | X | | | | | | | |
| Demographics | X | | | | | | | |
| Vital signs^{e} | X | X | X | X | X | X | | |
| Physical examination^{f} | X | | | | | | | |
| Body weight^{g} | X | X | | X | | X | | |
| 12-Lead ECG | X | X | | X | | X | | |
| Standard ophthalmologic | | X^{i, j} | | | | X | | |
| examination^{h} | | | | | | | | |
| Corneal specular microscopy^{h} | | Xⁱ | | | | | | X |
| Prior/concomitant | X | X | X | X | X | X | X | |
| medication/therapy | | | | | | | | |
| Randomization | | X | | | | | | |
| Clinical laboratory tests | X | X | X | X | X | X | | |
| (hematology, chemistry, urinalysis) | | | | | | | | |
| Serum/urine pregnancy test^{k} | X | X | X | X | X | X | | |
| UDS (onsite and central laboratory) | X | X | X | X | X | X | | |
| and breathalyzer alcohol screen | | | | | | | | |
| Blood sample for PK^{l} | | | | X | | X | | |
| Blood sample for genetic analysis | | X | | | | | | |
| and biomarker (LAC) analysis | | | | | | | | |
| (stored)^{m} | | | | | | | | |
| Thyroid function tests | X | | | | | | | |
| Viral serology | X | | | | | | | |
| Hepatic safety testingⁿ | [symbol] | | | | | | | |
| SCID-5-CT | X | | | | | | | |
| CGI-S | X | X | | | | X | | |
| CGI-I | | X | X | X | X | X | | |
| C-SSRS^{o} | X | X | X | X | X | X | X | |
| HAMD-17 | X | X | | X | | X | | |
| HAM-A | X | X | | X | | X | | |
| STAI | | X | | | | X | | |
| SDS | | X | | X | X | X | | |
| HADS | | X | X | X | X | X | | |
| SHAPS | X | X | | X | | X | | |
| PRT^{m} | | X | | | | X | | |
| PHQ-9 | X | X | | X | | X | | |
| Facial and speech recording | X | | | | | X | | |
| COWS | X | X | X | X | X | X | | |
| Adverse events^{p} | | X | X | X | X | X | X | |
| Complete IWRS | X | X | X | X | X | X | | |
| Dispense study drug | | X^{q} | X^{q} | X^{q} | X^{q} | | | |
| Study drug administration | | X^{r} | | X^{s} | | | | |
| Study drug compliance | | | X | X | X | X | | |

BMI = body mass index;
CRF = case report form;
CGI-I = Clinical Global Impression of Improvement;
CGI-S = Clinical Global Impression of Severity;
COWS = Clinical Opiate Withdrawal Scale;
C-SSRS = Columbia Suicide Severity Rating Scale;
d = day;
DSM = Diagnostic and Statistical Manual of Mental Disorders;
ECG = electrocardiogram;
eCRF = electronic case report form;
EOT = End of Treatment;
HADS = Hospital Anxiety and Depression Scale;
HAM-A = Hamilton Anxiety Rating Scale;
HAMD-17 = Hamilton Rating Scale for Depression - 17-Item Version;
IWRS = Interactive Web-response System;
LAC = acetyl-L-carnitine;
NA = not applicable;
PHQ-9 = Patient Health Questionnaire-9;
PK = pharmacokinetic;
PRT = Probabilistic Reward Task;
SDS = Sheehan Disability Scale;
SCID-5-CT = Structured Clinical Interview for DSM-5 Disorders, Clinical Trials Version;
SHAPS = Snaith-Hamilton Pleasure Scale;
STAI = State-Trait Anxiety Inventory;
TC = telephone call;
UDS = urine drug screen

Study site personnel contacted subjects by telephone for Safety Follow-up assessments (adverse events, concomitant medication usage, and C-SSRS) approximately 2 weeks after discontinuation of study drug.

Visit 1 procedures were completed over 2 days, when necessary.

Subjects who discontinued early, completed an EOT visit <2 days after their last dose of study drug; efficacy assessments were performed only if the subject had remained on study drug. For subjects who did not return, the site completed a C-SSRS if they were aware of a potential suicide-related thought or behavior by other communications. The 12-week corneal specular microscopy examination was still conducted after approximately 12 weeks post initial study drug exposure.

Inclusion/exclusion criteria were reconfirmed for eligibility prior to randomization.

Vital signs measurements (respiratory rate first, then blood pressure and pulse) were collected after subject had been supine for 10 minutes; subjects then stood for 3 minutes before obtaining standing vital signs measurements. Oral temperature was assessed in any position.

A full physical examination involving an evaluation of cardiovascular, respiratory, gastrointestinal, neurological (examination of cranial nerves, motor system, sensory, and reflexes), dermatological, and musculoskeletal systems, and included general appearance, skin, head, neck, eyes, ears, nose, throat, lungs, heart, abdomen, back, lymph nodes, extremities, and vascular status was conducted at the screening visit. Further physical examination was conducted as needed.

At Screening, height was collected for BMI calculation.

Ophthalmologic examinations were performed by a site-affiliated ophthalmologist. Details for the standard ophthalmologic examination and corneal specular microscopy were described in a separate ocular monitoring manual. The final ocular examination for corneal specular microscopy was conducted approximately 12 weeks after the first dose of study drug; exceptions were discussed with medical monitor.

Baseline ophthalmologic examinations (standard and corneal specular microscopy) were performed before randomization.

Prior to Visit 3 (Week 2) (between Days 7 and 14 [+2 days]), a standard ophthalmologic examination was performed.

Serum pregnancy test was performed at screening visit only and urine pregnancy tests were performed by the site at all subsequent visits.

At Visit 4, blood samples were collected from all subjects, pre-dose and 4 hours±1 hour post dose. At this visit, subjects took their study drug in the clinic with a light snack at the site. If a subject discontinued before completing the study drug treatment, every effort was made to obtain a blood sample at the final visit. In addition, a steady state blood sample was collected at Visit 6 (EOT); time of last dose of study drug was recorded.

Optional assessment; subject refusal to perform did not prohibit study participation.

Unscheduled hepatic monitoring testing was performed at any time on the basis of clinical laboratory testing results.

The C-SSRS Baseline version was used at Screening and “Since Last Visit” version was used for all subsequent visits.

Adverse events reported before first dose of study drug were recorded as medical history. Serious AEs were recorded on the AE eCRF from the time of informed consent.

Subjects were instructed to take their study drug daily in the morning and preferably with food.

Subjects took their first dose of study drug (the Study Compound or placebo) under the supervision of clinical study personnel during the baseline visit.

At Visit 4, study drug was taken at the study site after collection of the pre-dose blood sample.

### Results

Administration of 1-(6-ethyl-8-fluoro-4-methyl-3-(3-methyl-1,2,4-oxadiazol-5-yl)quinolin-2-yl)-N-(tetrahydro-2H-pyran-4-yl)piperidin-4 amine, as described herein, resulted in statistically significant reductions of anhedonia compared with placebo following 8 weeks of treatment in patients with moderate-to-severe MDD. These findings demonstrate that this compound is a novel monotherapy treatment option for patients with MDD beyond the currently approved agents.

For example, 204 patients were randomized and included in the safety population and 171 patients (1-(6-ethyl-8-fluoro-4-methyl-3-(3-methyl-1,2,4-oxadiazol-5-yl)quinolin-2-yl)-N-(tetrahydro-2H-pyran-4-yl)piperidin-4 amine, n=88; placebo, n=83) were included in the final efficacy population. A pre-specified last observation carried forward (LOCF) sensitivity analysis was used (as missingness exceeded 10%). There were significant and clinically-meaningful improvements in anhedonia, as assessed by the SHAPS, in this broad (mild-to-severe) MDD patient population (LSM CfB −7.8 vs −4.4, p=0.002). In a pre-specified subgroup analysis in patients with moderate-to-severe MDD (baseline HAMD-17≥22, n=100), 1-(6-ethyl-8-fluoro-4-methyl-3-(3-methyl-1,2,4-oxadiazol-5-yl)quinolin-2-yl)-N-(tetrahydro-2H-pyran-4-yl)piperidin-4 amine demonstrated more statistically significant and clinically meaningful improvements in anhedonia (LSM CfB −9.2 vs −4.4; p=0.001). The compound also demonstrated statistically significant and clinically meaningful improvements in depression as measured by HAMD-17 in the broad MDD population (LSM −8.9 vs −6.7, p=0.02) with magnified treatment effects in the moderate-to-severe (HAMD-17≥22) MDD population (LSM −9.9 vs −7.1, p=0.037). The compound was well tolerated with a safety profile comparable to placebo. The incidence of treatment-emergent adverse events (TEAEs) in the treatment group was 35.3% vs the placebo group at 44.1%. Most TEAEs were mild to moderate, with no severe TEAEs reported in the treatment group vs 4.9% in the placebo group. Discontinuation rates due to TEAEs in patients receiving NIRA-140 were 1.0% vs 11.8% in those receiving placebo. The compound was not associated with weight gain or sexual dysfunction and no evidence of increased suicidal ideation or behavior was identified.

LIST OF ABBREVIATIONS

| Abbreviation | |
|---|---|
| or Term | Definition |
| Ab | Antibody |
| ADD | attention deficit disorder |
| ADHD | attention deficit hyperactivity disorder |
| AE | adverse event |
| ALT | alanine aminotransferase |
| APA | American Psychiatric Association |
| AESI(s) | adverse event(s) of special interest |
| AST | aspartate aminotransferase |
| ANCOVA | analysis of the covariance |
| AUD | alcohol use disorder |
| BMI | body mass index |
| BP | blood pressure |
| CAT | Clinical Assessment Technology |
| CFR | Code of Federal Regulations |
| cGCP | current Good Clinical Practice |
| CGI-I | Clinical Global Impression of Improvement |
| CGI-S | Clinical Global Impression of Severity |
| CI | confidence interval |
| CIOMS | Council for International Organizations of Medical |
| | Sciences |
| CKD-EPI | Chronic Kidney Disease Epidemiology Collaboration |
| CNS | central nervous system |
| COVID-19 | coronavirus disease 2019 |
| COWS | Clinical Opiate Withdrawal Scale |
| CREB | cAMP response binding protein |
| CRF | case report form |
| CRO | Contract Research Organization |
| C-SSRS | Columbia Suicide Severity Rating Scale |
| CYP | cytochrome P450 |
| DSM-5 | Diagnostic and Statistical Manual of Mental Disorders |
| | Fifth Edition |
| DSM-IV-TR | Diagnostic and Statistical Manual of Mental Disorders, |
| | Fourth Edition, Text Revision |
| EC | Ethics Committee |
| ECG | electrocardiogram, electrocardiogra |
| eCRF | electronic case report form |
| EDC | electronic data capture |
| eGFR | estimated glomerular filtration rate |
| FDA | Food and Drug Administration |
| GAD | generalized anxiety disorder |
| GEE | generalized estimating equations |
| HADS | Hospital Anxiety and Depression Scale |
| HADS-A | Hospital Anxiety and Depression Scale - Anxiety |
| | Subscale |
| HADS-D | Hospital Anxiety and Depression Scale - Depression |
| | Subscale |
| HAM-A | Hamilton Anxiety Rating Scale |
| HAMD-17 | Hamilton Depression Rating Scale - 17-item Version |
| HAV | hepatitis A virus |
| HAV-Ab (IgM) | hepatitis A virus antibody |
| HBs Ag | hepatitis B surface antigen |
| HCV | hepatitis C virus |
| HIPAA | Health Insurance Portability and Accountability Act |
| HIV | human immunodeficiency virus |
| HPMC | hydroxypropyl methylcellulose |
| IC50 | half-maximal inhibitory concentration |
| ICC | intraclass correlation coefficient |
| ICF | informed consent form |
| ICH | International Council for Harmonisation of Technical |
| | Requirements for Pharmaceuticals for Human Use |
| IEC | Independent Ethics Committee |
| IgM | immunoglobulin M |
| IND | US Investigational New Drug Application |
| IRB | Institutional Review Board |
| IWRS | interactive web-response system |
| KOR | kappa opioid receptor |
| KORA | kappa opioid receptor antagonist |
| LAC | acetyl-L-carnitine |
| LC-MS/MS | liquid chromatography-tandem mass spectrometry |
| LLN | lower limit of normal |
| LS Means | least-square means |
| MedDRA | Medical Dictionary for Regulatory Activities |
| MDD | major depressive disorder |
| MMRM | mixed-models repeated measures |
| NOAEL | no-observed-adverse-effect-level |
| OPRK1 | opioid receptor kappa 1 |
| PDF | portable document format |
| PET | positron emission tomography |
| P-gp | P-glycoprotein |
| PHQ-9 | Patient Health Questionnaire-9 |
| PK | pharmacokinetic(s) |
| POC | proof of concept |
| PP | per-protocol |
| PRT | probabilistic reward task |
| PTSD | post-traumatic stress disorder |
| QD | once daily |
| QTcF | Fridericia's correction method for QT Interval |
| REB | Research Ethics Board |
| RO | receptor occupancy |
| S-anxiety | STAI state anxiety subscale score |
| SAE | serious adverse event |
| SAP | statistical analysis plan |
| SCID-5-CT | Structured Clinical Interview for DSM-5 Disorders, |
| | Clinical Trials Version |
| SD | standard deviation |
| SDS | Sheehan Disability Scale |
| SHAPS | Snaith-Hamilton Pleasure Scale |
| SIGH-A | Structured Interview Guide for the Hamilton Anxiety |
| | Rating Scale |
| SIGH-D | Structured Interview Guide for the Hamilton |
| | Depression Rating Scale |
| SNRI | serotonin norepinephrine reuptake inhibitor |
| SOP | Standard Operating Procedure |
| SSRI | selective serotonin reuptake inhibitor |
| STAI | State-Trait Anxiety Inventory |
| SUSAR | serious unexpected suspected adverse reaction |
| T-anxiety | STAI trait anxiety subscale score |
| TC | telephone call |
| TEAE | treatment-emergent adverse event |
| THC | Tetrahydrocannabinol |
| TPO | third-party organization |
| TSH | thyroid-stimulating hormone |
| UDS | urine drug screen |
| ULN | upper limit of normal |
| US | United States |
| WHO | World Health Organization |
| WHODrug | World Health Organization Drug classification |

A number of embodiments of the present disclosure have been described. Nevertheless, it will be understood that various modifications may be made without departing from the spirit and scope of the disclosure. Accordingly, other embodiments are within the scope of the following claims.

1. A method of treating anhedonia in a subject in need thereof, comprising administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof.

2. (canceled)

3. The method of claim 1,
wherein the subject in need thereof has been previously identified or diagnosed as having depression.

4.-8. (canceled)

9. The method of claim 3, wherein the depression is major depressive disorder (MDD) or treatment resistant depression (TRD).

10.-11. (canceled)

12. The method of claim 3, wherein the depression further comprises anxiety.

13. The method of claim 1, wherein the subject has a Snaith-Hamilton Pleasure Scale (SHAPS) score of from 31 to 56 prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof.

14.-16. (canceled)

17. The method of claim 13, wherein the SHAPS score of the subject is decreased by about 2 to about 4 after four weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof; the SHAPS score of the subject is decreased by about 4 to about 6 after eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof; or both.

18.-20. (canceled)

21. A method of treating anhedonia in a subject in need thereof, comprising:
(a) determining a first SHAPS score in the subject;
(b) administering to the subject a therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof for a period of time; and
(c) determining a second SHAPS score in the subject; wherein the second SHAPS score is less than the first SHAPS score.

22. The method of claim 21, wherein the period of time is about 1 week to about 52 weeks, about 4 weeks to about 26 weeks, about 4 weeks to about 12 weeks, or about 4 weeks to about 8 weeks.

23.-25. (canceled)

26. The method of claim 21, wherein
the first SHAPS score is from 31 to 56;
the second SHAPS score is lower than the first SHAPS score by about 2 to about 5; or
the first SHAPS score is from 31 to 56 and the second SHAPS score is lower than the first SHAPS score by about 2 to about 5.

27.-28. (canceled)

29. The method of claim 21, wherein the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof is administered once daily for the period of time.

30. The method of claim 1, wherein the subject has previously been administered one or more antidepressant medications.

31. The method of claim 30, wherein the subject did not exhibit a clinically meaningful response to the one or more previously administered antidepressant medications.

32. The method of claim 30, wherein the one or more previously administered antidepressant medications are selected from selective serotonin reuptake inhibitors, selective norepinephrine reuptake inhibitors, and kappa opioid receptor antagonists.

33.-36. (canceled)

37. The method of claim 32, wherein the kappa opioid receptor antagonist is aticaprant.

38. The method of claim 1, wherein the subject has not been previously administered one or more antidepressant medications.

39. The method of claim 1, wherein the subject has not been previously administered a selective serotonin reuptake inhibitor.

40. The method of claim 1, wherein the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof is from about 20 mg/day to about 160 mg/day, from about 20 mg/day to about 80 mg/day, from about 80 mg/day to about 120 mg/day, or from about 120 mg/day to about 160 mg/day.

41.-43. (canceled)

44. The method of claim 1, wherein the therapeutically effective amount of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof is 40 mg/day, 80 mg/day, 120 mg/day, or 160 mg/day.

45.-47. (canceled)

48. The method of claim 1, wherein the subject has not had a score of “YES” on C-SSRS Item 4 or Item 5 within 3 months prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof.

49. The method of claim 1, wherein the subject has a HAMD-6 Total Score of from 11 to 22, a HAMD-17 Total Score of 22 to 52, or both, prior to the first administration of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof.

50. The method of claim 49, wherein the HAMD-6 Total Score of the subject is decreased by about 1 to about 3 after four weeks or eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof.

51.-52. (canceled)

53. The method of claim 49, wherein the HAMD-17 Total Score of the subject is decreased by about 2 to about 4 after four weeks or eight weeks of once daily administration of 1-[6-ethyl-8-fluoro-4-methyl-3-(3-methyl-1,2,4-oxadiazol-5-yl)quinolin-2-yl]-N-(oxan-4-yl)piperidin-4-amine or a pharmaceutically acceptable salt thereof.

54. (canceled)

55. The method of claim 1, wherein the 1-[6-ethyl-8-fluoro-4-methyl-3-(3-methyl-1,2,4-oxadiazol-5-yl)quinolin-2-yl]-N-(oxan-4-yl)piperidin-4-amine is administered as the free base or as a pharmaceutically acceptable salt.

56. (canceled)

57. The method of claim 1, wherein the subject is a human.
